(19) United States
(12) Patent Application Publication
(10) Pub. No.: US 2024/0344013 A1
(43) Pub. Date: Oct. 17, 2024

(54) METABOLOMIC ANALYSIS

(71) Applicant: Genentech, Inc., South San Francisco, CA (US)

(72) Inventors: Zijuan LAI, South San Francisco, CA (US); Shahram MISAGHI, Oakland, CA (US); Dewakar SANGARAJU, South San Francisco, CA (US); Danming TANG, Foster City, CA (US)

(73) Assignee: Genentech, Inc., South San Francisco, CA (US)

(21) Appl. No.: 18/604,429

(22) Filed: Mar. 13, 2024

Related U.S. Application Data
(63) Continuation of application No. PCT/US2022/043652, filed on Sep. 15, 2022.
(60) Provisional application No. 63/244,609, filed on Sep. 15, 2021.

(51) Int. Cl. C12M 1/34; C12M 1/36; G01N 30/72

(52) U.S. Cl. CPC C12M 41/38; C12M 41/48; G01N 30/72

(57) ABSTRACT

Provided herein are methods for quantifying the concentration of multiple metabolites in a sample. Also provided are methods for relative quantification of multiple metabolites in a sample, methods for monitoring the course of a cell culture, and methods for optimising a cell culture.

### CROSS-REFERENCE TO RELATED APPLICATION

This application claims the benefit of U.S. Provisional Application No. 63/244,609, filed on Sep. 15, 2021, which is hereby incorporated by reference in its entirety.

### BACKGROUND

This invention relates to methods for quantifying the concentration of multiple metabolites in a sample. Also provided are methods for relative quantification of multiple metabolites in a sample, methods for monitoring the course of a cell culture, and methods for optimising a cell culture.

Fed-batch culture is a commonly used cell culture mode in the field of biotechnology and bioengineering to maximize titer and volumetric productivity for recombinant protein manufacturing. In the production process of fed-batch culture, glucose and amino acids are major nutrition sources in chemically defined production media. The cells typically consume those nutrients to a significant extent for energy metabolism and recombinant protein production. This does, however, result in the generation and accretion of toxic metabolic waste products (R. P. Nolan and K. Lee, Metabolic engineering, 2011, 13, 108-124). Lactate and ammonia are two metabolic byproducts, mainly from glucose metabolism, that are known to accumulate in cell fed-batch cultures. These two well-known metabolic waste products adversely impact the growth of mammalian cell lines, as well as productivity and glycosylation pattern of protein biologics. More recently, the cell culture community has been investing in understanding more metabolic byproducts beyond lactate and ammonia. Several research groups have shown that CHO cells in fed-batch cultures produce various metabolic intermediates (e.g. isobutyric acid and isovaleric acid) from amino acid metabolism (S. Pereira, H. F. Kildegaard and M. R. Andersen, Biotechnology journal, 2018, 13, 1700499), and that these byproducts inhibit cellular growth and antibody drug productivity to varying degrees at accumulated concentrations (N. Carinhas, T. M. Duarte, L. C. Barreiro, M. J. Carrondo, P. M. Alves and A. P. Teixeira, Biotechnology and bioengineering, 2013, 110, 3244-3257; B. C. Mulukutla, J. Kale, T. Kalomeris, M. Jacobs and G. W. Hiller, Biotechnology and bioengineering, 2017, 114, 1779-1790; B. C. Mulukutla, J. Mitchell, P. Geoffroy, C. Harrington, M. Krishnan, T. Kalomeris, C. Morris, L. Zhang, P. Pegman and G. W. Hiller, Metabolic engineering, 2019, 54, 54-68).

Significant research efforts have been devoted towards identification and regulation of novel inhibitory metabolites in cell fed-batch cultures using modern omics technologies, such as genomics, transcriptomics, proteomics, and metabolomics analysis. Essentially, cell metabolism is the main driver that causes remarkable changes in the growth environment and further affects productivity and product quality. Transcriptomics complemented with proteomics for comprehensive profiling of expressed gene transcripts and enzymatic proteins have developed a better understanding towards metabolic homeostasis in cell culture bioprocesses (P. Datta, R. J. Linhardt and S. T. Sharfstein, Biotechnology and bioengineering, 2013, 110, 1255-1271; H. F. Kildegaard, D. Baycin-Hizal, N. E. Lewis and M. J. Betenbaugh, Current opinion in biotechnology, 2013, 24, 1102-1107). Among a broad range of omics techniques, contemporary metabolomics analysis has enabled us to directly identify key metabolites and metabolic pathways closely associated with growth inhibition and productivity limitation (W. P. Chong, F. N. Yusufi, D.-Y. Lee, S. G. Reddy, N. S. Wong, C. K. Heng, M. G. Yap and Y. S. Ho, Journal of biotechnology, 2011, 151, 218-224; S. Selvarasu, Y. S. Ho, W. P. Chong, N. S. Wong, F. N. Yusufi, Y. Y. Lee, M. G. Yap and D. Y. Lee, Biotechnology and bioengineering, 2012, 109, 1415-1429). Quantitative monitoring of inhibitory metabolites in production media could contribute to actively tracking and deciphering cellular phenotype, as well as acting as potential prediction index for cell viability (N. Alden, R. Raju, K. McElearney, J. Lambropoulos, R. Kshirsagar, A. Gilbert and K. Lee, Metabolites, 2020, 10, 199).

As discovery and control of inhibitory mechanisms may be useful in improving cell culture performance during antibody production process, there is a need to develop a targeted and high throughput analytical method for more reliable, robust and prompt quantification of growth inhibition related metabolites in cell fed-batch cultures.

### BRIEF SUMMARY OF THE DISCLOSURE

In accordance with the present invention there is provided a method for quantifying the concentration of multiple metabolites. Such methods may include relative quantification or absolute quantification. Also provided are methods of monitoring the course of a cell culture, e.g. by determining and comparing the levels of multiple metabolites at different time points; as well as methods of optimising a cell culture, e.g. by adjusting a cell culture condition in response to a change in concentration of at least one metabolite.

A first aspect of the invention provides method for quantifying the concentration of multiple metabolites in a sample, wherein each metabolite comprises of at least one carbonyl group, the method comprising:
(a) adding a known amount of an internal standard (optionally a stable isotope labelled analogue) corresponding to each of the multiple analytes of the sample;
(b) contacting the sample with a reagent comprising a carbonyl reactive group and a masking group, thereby derivatising the carbonyl group of each metabolite with the masking group and providing a derivatised sample;
(c) subjecting the derivatised sample to chromatographic separation and full scan accurate mass high resolution mass spectrometry; and
(d) quantifying the amount of each of the multiple metabolites based on a monoisotopic signal obtained for said metabolite,
wherein said quantifying comprises comparing the monoisotopic signal obtained for each said one of the multiple metabolites to an external calibration for said one of the multiple metabolites, after normalising the external calibration using the signal obtained for the known amount of the stable isotope labelled analogue corresponding to the said one of the multiple metabolites; and wherein the monoisotopic signal obtained for said metabolite corresponds to the most abundant positive ion isotopologue, unless said most abundant positive ion isotopologue has a signal at or above the upper limit of the external calibration for said metabolite, in which case the monoisotopic signal obtained for said metabolite corresponds to a less abundant positive ion isotopologue of said metabolite.

A second aspect of the invention provides a method for relative quantification of multiple metabolites in a sample, wherein each metabolite comprises at least one carbonyl group, the method comprising
(a) contacting the sample with a reagent comprising a carbonyl reactive group and a masking group, thereby derivatising the carbonyl group of each metabolite with the masking group and providing a derivatised sample;
(b) subjecting the derivatised sample to chromatographic separation and full scan accurate mass high resolution mass spectrometry; and
(c) quantifying the amount of each of the multiple metabolites based on a monoisotopic signal obtained for each said metabolite,
wherein said quantifying comprises comparing the monoisotopic signal obtained for each said one of the multiple metabolites to the monoisotopic signal obtained for each said other of the multiple metabolites, thereby obtaining the relative quantification, wherein the monoisotopic signal obtained for each one of the metabolites corresponds to the most abundant positive ion isotopologue, unless said most abundant positive ion isotopologue has a signal at or above the upper limit of quantification for said metabolite, in which case the monoisotopic signal obtained for said metabolite corresponds to a less abundant positive ion isotopologue of said metabolite.

A third aspect of the invention provides a method of monitoring the course of a cell culture, comprising:
(a) performing a method of the first aspect or second aspect at a first time point, wherein the sample is a sample of a cell culture;
(b) repeating the method of the first aspect or second aspect at a second time point; and
(c) optionally repeating the method of the first aspect or second aspect at one or more subsequent time points,
   wherein the monitoring comprises comparing the levels of each of the multiple metabolites between each of the multiple time points.

A fourth aspect of the invention provides a method of optimising a cell culture, comprising:
(a) performing a method of the first aspect or second aspect at a first time point, wherein the sample is a sample of a cell culture;
(b) repeating a method of said one of the first or second aspects at a second time point; and
(c) optionally repeating a method of said one of the first or second aspects at one or more subsequent time points,
   and, in response to a change in the concentration of at least one of the multiple metabolites between the first time point and a second or subsequent time point, adjusting a cell culture condition.

### BRIEF DESCRIPTION OF THE DRAWINGS

Embodiments of the invention are further described hereinafter with reference to the accompanying drawings, in which:

FIG. 1 indicates the steps in a generalised method for quantifying the concentration of multiple metabolites in a sample. FIG. 1 includes sampling step 1, extraction step 2, derivatization step 3, chromatographic analysis step 4, and data analysis step 5.

FIG. 2 is a schematic of a method of optimising a cell culture.

FIG. 3 shows the results of CHO cells that were initially cultured to inoculate the N−1 stage of the inoculum train followed by transfer to the production (N) vessel with different groups (C1-C7, as defined in Table 3) at varied inhibitor levels. A) Cell growth and viability at the N−1 stage. B) High level of inhibitory metabolites at N−1 stage of the inoculum train correlated with lower cell growth rate in production (N) stage. C) Titer data from a short (3 day) production culture shows that cells derived from all different N−1 stages are capable of antibody expression in early phases of production culture and that accumulation of growth inhibitory metabolites does not interfere with antibody expression in these cells. D) Isovalerate accumulation at N−1 stage is above the reported inhibitory level* after day 4 in culture. E) Isovalerate accumulation at production N stage. F) Formate accumulation at N−1 stage is above the reported inhibition level* after day 3 in culture. G) Formate accumulation at production N stage. *As reported in B. C. Mulukutla, J. Kale, T. Kalomeris, M. Jacobs and G. W. Hiller, Biotechnology and bioengineering, 2017, 114, 1779-1790.

FIG. 4 shows an exemplary derivatization step used in methods of the invention, wherein carbonyl (e.g. carboxyl or ketone) containing compounds are modified through EDC activation and O-BHA derivatization to produce amides (in the case of carboxyl) and oximes (in the case of ketones).

FIG. 5 shows characteristic LC-HRMS chromatograms of target metabolites in CHO cell fed-batch cultures. Extracted ion chromatograms were generated using protonated target ions with 5 ppm mass accuracy. LC-HRMS peaks with * notation indicate isomeric ions in the matrix with no interference to the analytes.

FIG. 6 shows one-way ANOVA (A) and Heat Map (B) to illustrate the statistical significance and global view of growth inhibition related metabolic changes in mAb production process. Metabolomics samples were acquired from four independent bioreactors (biological replicates in Heat Map) on day 1, day 6, and day 11 to represent the CHO cell growth phase, stationary phase, and death phase, respectively. Significance threshold was set as p<0.05 and values were expressed as −Log 10(p) format for all target analytes. Metabolite abbreviations marked on ANOVA plot are explained with complete compound names on Heat Map.

FIG. 7 provides scattered boxplots that demonstrate concentrations of 21 target metabolites in CHO cell fed-batch cultures during antibody production process. Metabolomics samples were acquired from four independent bioreactors (biological replicates in boxplots) on day 1, day 6, and day 11 to represent the CHO cell growth phase, stationary phase, and death phase, respectively. Meaning of color notation and statistical significance symbol, as well as cell viability information are shown on the right side of the figure.

FIG. 8 provides a multivariate statistical analysis—PCA and PLS-DA to characterize cellular stages in mAb production process. A: Principal Component Analysis (PCA) clearly indicated that production cell culture samples were well classified. B: Partial Least Squares Discriminant Analysis (PLS-DA) through VIP (Variable Importance in Projection) score ranking resulted Top 10 metabolic features to differentiate cellular stages.

FIG. 9 indicates the amino acid metabolic sources for exemplary metabolites of interest in CHO cell fed-batch cultures. The Top values marked next to target analytes referred to their VIP score ranking numbers in PLS-DA analysis. Metabolic multistep reaction mechanisms are detailed in KEGG (https://www.genome.jp/kegg/pathway.html) biological pathway database.

FIG. 10 illustrates the relative concentration trends from day 2 to day 7 for metabolites formic acid, butyric acid, isobutyric acid, isovaleric acid, caproic acid, 2-methylbutyric acid, 2-hydroxyisovaleric acid, 2-hydroxyisocaproic acid, indole-3-acetic acid, indole-3-lactic acid, phenylacetic acid, phenyllactic acid, 4-hydroxyphenylacetic acid and 4-hydroxyphenyllactic acid.

### DETAILED DESCRIPTION

The abbreviations used herein have their conventional meaning within the chemical and biological arts.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

For the avoidance of doubt, it is hereby stated that the information disclosed earlier in this specification under the heading “Background” is relevant to the invention and is to be read as part of the disclosure of the invention.

All publications, patent applications, patents, and other references mentioned herein are incorporated by reference in their entirety. In case of conflict, the present specification, including definitions, will control.

### Definitions

The following explanations of terms and methods are provided to better describe the present disclosure and to guide those of ordinary skill in the art in the practice of the present disclosure.

The term “about” or “approximately”, unless otherwise stated, means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “on-line”, unless otherwise stated, refers to methods where the sampling and analysis of samples are performed in an automated manner. It also refers to apparatus adapted to perform such methods. For example, in an on-line method a liquid may be automatically sampled and provided in fluid communication to an analyser. Such on-line systems may use, for example, autosamplers, valves, loops, cartridges and/or columns in the fluid path between sampling and analyser. Apparatus adapted to perform on-line methods may comprise a controller programmed to perform said on-line methods. “Off-line” methods differ from on-line methods in that the sampling step is not provided in fluid communication with the analyser. For example, in an off-line method a sample may be obtained and then manually placed in an autosampler, or a sample may be obtained and then manually introduced into an analyser (for example by injection or infusion).

The term “liquid chromatography” (LC), unless otherwise stated, refers to a separation technique in which molecules or ions of interest are separated by differential partitioning between a stationary phases and a mobile liquid phase. The term LC, unless the context requires otherwise, comprises high performance liquid chromatography (HPLC), ultra performance liquid chromatography (UPLC), hydrophilic interaction liquid chromatography (HILIC), reverse phase liquid chromatography (e.g. using C18, C8 or phenyl-hexyl columns), and the like.

The terms “polypeptide” and “protein” can be used interchangeably and refer generally to peptides and proteins having more than about 10 covalently attached amino acids linked by a peptidyl bond. The term protein encompasses purified natural products, or products which may be produced partially or wholly using recombinant or synthetic techniques. The terms peptide and protein may refer to an aggregate of a protein such as a dimer or other multimer, a fusion protein, a protein variant, or derivative thereof. The term also includes modifications of the protein, for example, protein modified by glycosylation, acetylation, phosphorylation, pegylation, ubiquitination, and so forth. A protein may comprise amino acids not encoded by a nucleic acid codon. A protein may have a sequence of amino acids of sufficient length to produce higher levels of tertiary and/or quaternary structure. A typical protein herein may have a molecular weight of at least about 15-20 kD, preferably at least about 20 kD. Examples of proteins encompassed within the definition herein include all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “carbonyl” as used herein includes reference to a carboxylic acid, a ketone and an aldehyde. Carbonyl may refer to a carboxylic acid or a ketone. Carbonyl may refer to a carboxylic acid. Carbonyl may refer to a ketone.

The term “cell” as used herein includes reference to a eukaryotic cell. Unless the context requires otherwise, reference to a cell may include reference to the plural (cells). A eukaryotic cell may be an animal cell (e.g. a mammalian cell) or a fungal cell (e.g. a yeast cell). A eukaryotic cell may be a mammalian cell, such as a hybridoma, CHO cell, COS cell, VERO cell, HeLa cell, HEK 293 cell, PER-C6 cell, K562 cell, MOLT-4 cell, MI cell, NS-1 cell, COS-7 cell, MDBK cell, MDCK cell, MRC-5 cell, WI-38 cell, WEHI cell, SP2/0 cell, BHK cell (including BHK-21 cell) and derivatives thereof. A CHO cell may be, for example, a CHO K1 cell, a CHO K1SV cell, a DG44 cell, a DUKXB-11 cell, a CHOK1S cell, a CHO K1M cell, and derivatives thereof. Derivatives of said cells may represent cells that are derived by natural evolution and/or genetic engineering from said cells, including those CHO cells engineered for targeted integration of a gene of interest (for e.g., as in WO2019126634, hereby incorporated by reference). Preferred cells are suitable for protein expression.

The term “cell line” as used herein includes reference to a culture of eukaryotic cells that can be propagated repeatedly. The eukaryotic cells of the cell line may be selected from any cell as defined herein.

The terms “host cell,” “host cell line” and “host cell culture” are used interchangeably herein to refer to cells into which exogenous nucleic acid has been introduced, including the progeny of such cells. Host cells include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein.

The terms “mammalian host cell” or “mammalian cell” as used herein refer to cells and cell lines derived from mammals that are capable of growth and survival when placed in either monolayer culture or in suspension culture in a medium containing the appropriate nutrients and growth factors. The necessary growth factors for a particular cell line are readily determined empirically without undue experimentation, as described for example in Mammalian Cell Culture (Mather, J. P. ed., Plenum Press, N.Y. 1984), and Barnes and Sato, (1980) Cell, 22:649. Typically, the cells are capable of expressing and secreting large quantities of a particular protein, e.g., glycoprotein, of interest into the culture medium. Examples of suitable mammalian host cells include Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989); CHO-K1 (ATCC, CCL-61); monkey kidney CV1 line transformed by SV40 (COS-7, ATCC CRL 1651); human embryonic kidney line (293 or 293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2). The mammalian cells may include Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989) or other CHO cell derivatives including but not limited to CHO K1, CHO K1SV, DG44, DUKXB-11 cell culture, CHOK1S cell culture, CHO K1M. The mammalian cells may also include CHO or CHO cell derivatives engineered for targeted integration of any gene of interest (for e.g., as in WO2019126634, hereby incorporated by reference).

The term “cell culture medium” as used herein refers to a nutritive solution for cultivating cells. A “cell culture feed” and a “cell culture additive” represent nutritive supplements that may be added to a cell culture medium to improve medium performance. For example, a cell culture feed and/or a cell culture additive may be added to a cell culture medium during batch culture of cells. A cell culture medium may be chemically defined or may comprise undefined components. Cell culture medium, for example for mammalian cells, typically comprises at least one component from one or more of the following categories:
1) an energy source, usually in the form of a carbohydrate such as glucose;
2) all essential amino acids, and usually the basic set of twenty amino acids plus cysteine;
3) vitamins and/or other organic compounds required at low concentrations;
4) free fatty acids; and
5) trace elements, where trace elements are defined as inorganic compounds or naturally occurring elements that are typically required at very low concentrations, usually in the micromolar range.

Cell culture media and similar nutrient solutions may optionally be supplemented with one or more components from any of the following categories:
1) hormones and other growth factors as, for example, insulin, transferrin, and epidermal growth factor;
2) salts and buffers as, for example, calcium, magnesium, and phosphate;
3) nucleosides and bases such as, for example, adenosine, thymidine, and hypoxanthine; and
4) protein and tissue hydrolysates.

The term “culturing” refers to contacting a cell or cells with a cell culture medium under conditions suitable to the survival and/or growth and/or proliferation of the cell. A “cell culture” refers to a cell or cells in contact with a cell culture medium.

The term “batch culture” refers to a culture in which all components for cell culturing (including the cells and all culture nutrients) are supplied to the culturing bioreactor at the start of the culturing process.

The term “fed batch cell culture,” as used herein refers to a batch culture wherein the cells and culture medium are supplied to the culturing bioreactor initially, and additional culture nutrients are fed, continuously or in discrete increments, to the culture during the culturing process, with or without periodic cell and/or product harvest before termination of culture.

The term “perfusion culture,” sometimes referred to as continuous culture, is a culture by which the cells are restrained in the culture by, e.g., filtration, encapsulation, anchoring to microcarriers, etc., and the culture medium is continuously, step-wise or intermittently introduced (or any combination of these) and removed from the culturing bioreactor.”

The term “cell lysate” refers to a fluid containing the contents of lysed cells. The cells may be lysed using any known process, for example chemical, acoustic or mechanical lysis. Preferably the cells are lysed after the cells have been separated from the cell culture medium.

The terms “expression” or “expresses” are used herein to refer to transcription and translation occurring within a host cell. The level of expression of a product gene in a host cell can be determined on the basis of either the amount of corresponding mRNA that is present in the cell or the amount of the protein encoded by the product gene that is produced by the cell. For example, mRNA transcribed from a product gene may be quantified by northern hybridization. Sambrook et al., Molecular Cloning: A Laboratory Manual, pp. 7.3-7.57 (Cold Spring Harbor Laboratory Press, 1989). Protein encoded by a product gene can be quantified either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989). The expressed protein's PTMs may be assessed using methods disclosed herein.

The term “product quality” as used herein with reference to a protein product refers to the post-translational modification of the protein product. A protein product of requisite product quality will have the desired levels of post-translational modification. Relevant post-translational modifications include glycosylation, charge variant, or other post-translational modifications (e.g. oxidation, acetylation, amino acid misincorporation, etc) that can be measured by conventional methods, such as by Raman spectroscopy, chromatography, or mass spectrometry.

The term “stable isotope labelled analogue” refers to a chemical species in which one or more atoms on the molecule have been replaced with a corresponding stable isotope to produce a species which can act as a tracer used to model chemical and biochemical systems. Examples include replacing ¹H with ²H (i.e. deuterium), replacing ¹²C with ¹³C, or replacing ¹⁴N with ¹⁵N.

The term “normalising” used herein in relation to the calibration curve refers to the process of computing the curve using a linear regression model between analyte concentration and peak area ratio of analyte as adjusted in view of the signal(s) obtained for the corresponding internal standards.

The term “isotopologues” as used herein refers to molecules or ions that differ from each other in that at least one atom of the molecules or ions has a different number of neutrons. For example, one or more ¹H may be replaced with one or more ²H; one or more ¹²C may be replaced with one or more ¹³C, or one or more ¹⁴N may be replaced with one or more ¹⁵N.

The term “positive ion isotopologue” means, unless the context requires otherwise an isotopologue molecule that is associated with a cation to provide a positive ion. The cation may be, for example, a proton (H⁺), Na⁺, K⁺, NH₄⁺. The positive ion isotopologue may be a protonated molecule.

The term “protonated molecule” as used herein in the context of the monoisotopic signal obtained for a metabolite refers to an ion formed by interaction of a molecule with a proton abstracted from an ion according to the reaction: M+XH⁺→MH⁺+X. The symbolism [M+H]⁺ may also be used to represent the protonated molecule.

The term “inhibitory metabolites” refers to compounds produced by cellular metabolism which cause inhibition of the growth of the cells in the cell culture as the concentration of the metabolites increases.

The term “relative quantification” as used herein refers to an embodiment of a method of quantifying the concentration of multiple metabolites in a sample which does not involve the addition of an internal standard to the sample. In relative quantification, the monoisotopic signal obtained for each of the metabolites is compared to the monoisotopic signal obtained for at least one other metabolite to produce a relative concentration, thereby avoiding the need for an internal standard.

The “upper limit of quantification” as used herein refers to the highest concentration in the calibration that can be determined with the required precision and accuracy. The required precision may be less than 20% and the accuracy may be not more than 15% of the theoretical value. Alternatively, the upper limit of quantification may correspond to the highest concentration in the calibration curve.

### Methods of Quantifying the Concentration of Multiple Metabolites

Provided herein are methods for the quantification of multiple metabolites in a sample. These methods provide various advantages. For example, the present methods allow rapid determination of metabolite levels. This may allow close to real-time (e.g. within 2 or so hours) determination of metabolite levels in a cell culture, allowing for timely adjustment of cell culture conditions in response to metabolite levels. This assists in optimising cell culture conditions and may assist in maximising product quantity and/or quality. For example, productivity and/or titre may be improved. Additionally, the methods of the invention help achieve a balance between cell proliferation, protein glycosylation, therapeutics production, and metabolic waste accumulation, thereby improving the production process of CHO cell fed-batch strategy.

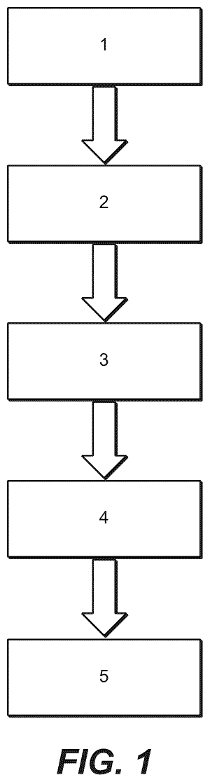

These methods for quantifying multiple metabolites in a sample typically follow the general approach set out in FIG. 1. FIG. 1 includes a sampling step 1, an extraction step 2, a derivatization step 3, a chromatographic analysis step 4, and a data analysis step 5. The first step, sampling step 1, may be performed in a bioreactor. The sampling step 1 may also include separation of cell culture media from the sample liquid. The second step, extraction step 2, comprises the extraction of metabolites from the separated cell culture. The third step, derivatization step 3, comprises reacting each extracted metabolite with a reagent comprising a reactive group and a masking group, thereby providing a derivatized metabolite sample. The derivatization step 3 may further include cleaning and drying the metabolite sample after performing the derivatization reaction(s). The fourth step, chromatographic analysis 4, comprises separation of the multiple metabolites in a chromatographic method followed by a mass spectrometry method. The chromatographic analysis 4 may comprise analysis by LC-MS (e.g. LC-HRMS). The fifth step, data analysis 5, comprises quantifying the amount of each metabolite based on a monoisotopic signal obtained for each metabolite. The data analysis may comprise comparing each monoisotopic signal to an external calibration curve. Alternatively, the data analysis may comprise comparing each monoisotopic signal to the monoisotopic signal obtained for another metabolite.

Where the data analysis comprises comparing each monoisotopic signal to an external calibration curve, each said calibration curve may comprise any suitable number of calibration points. For example, an external calibration curve may comprise at least 2 or 3 points, e.g., at least 4, 5, 6, 7, 8, 9 or 10 calibration points. For example, a calibration curve may comprise 10 calibration points.

An embodiment provides a method for quantifying the concentration of multiple metabolites in a sample, wherein each metabolite comprises of at least one carbonyl group, the method comprising: (a) adding a known amount of an internal standard (optionally a stable isotope labelled analogue) corresponding to each of the multiple analytes of the sample; (b) contacting the sample with a reagent comprising a carbonyl reactive group and a masking group, thereby derivatising the carbonyl group of each metabolite with the masking group and providing a derivatised sample; (c) subjecting the derivatised sample to chromatographic separation and full scan accurate mass high resolution mass spectrometry; and (d) quantifying the amount of each of the multiple metabolites based on a monoisotopic signal obtained for said metabolite, wherein said quantifying comprises comparing the monoisotopic signal obtained for each said one of the multiple metabolites to an external calibration for said one of the multiple metabolites, after normalising the external calibration using the signal obtained for the known amount of the stable isotope labelled analogue corresponding to the said one of the multiple metabolites; and wherein the monoisotopic signal obtained for said metabolite corresponds to the most abundant positive ion isotopologue, unless said most abundant positive ion isotopologue has a signal at or above the upper limit of the external calibration for said metabolite, in which case the monoisotopic signal obtained for said metabolite corresponds to a less abundant positive ion isotopologue of said metabolite.

The at least one carbonyl group may be selected from a carboxylic acid, a ketone and an aldehyde. The at least one carbonyl group may be a carboxylic acid or a ketone. The at least one carbonyl group may be a carboxylic acid. The at least one carbonyl group may be a ketone.

The carbonyl reactive group may be selected from a carboxyl acid reactive group, a ketone reactive group and an aldehyde reactive group. The carbonyl reactive group may be a carboxylic acid reactive group or a ketone reactive group. The carbonyl reactive group may be a carboxylic acid reactive group. The carbonyl reactive group may be a ketone reactive group.

Preferably, the at least one carbonyl group is a carboxylic acid and the carbonyl reactive group is a carboxylic acid reactive group. Alternatively, the at least one carbonyl group is a ketone and the carbonyl reactive group is a ketone reactive group.

The sample may be a sample of a cell culture. For example, the sample may be a cell culture medium and/or the sample may be a cell culture medium of cell lysate.

In embodiments, the sample is a cell culture medium. The cell culture may be an animal cell culture, e.g. a mammalian cell culture or an insect cell culture. Alternatively, the cell culture may be a fungal cell culture, e.g. a yeast cell culture. The cell culture may be a prokaryotic cell culture, e.g. an E. coli cell culture.

The cell culture may be a hybridoma culture, CHO cell culture, COS cell culture, VERO cell culture, HeLa cell culture, HEK 293 cell culture, PER-C6 cell culture, K562 cell culture, MOLT-4 cell culture, MI cell culture, NS-1 cell culture, COS-7 cell culture, MDBK cell culture, MDCK cell culture, MRC-5 cell culture, WI-38 cell culture, WEHI cell culture, SP2/0 cell culture, or BHK cell culture (including BHK-21 cell), or any derivative thereof.

The cell culture may be a CHO cell culture. In embodiments, the CHO cell culture is a CHO K1 cell culture, a CHO K1SV cell culture, a DG44 cell culture, a DUKXB-11 cell culture, a CHOK1S cell culture, or a CHO K1M cell culture, or a derivative thereof. Derivatives of said CHO cell culture may comprise CHO K1, CHO K1SV, DG44, DUKXB-11, CHOK1S, or any CHO cell or CHO derivative cell engineered for targeted integration of a gene of interest from said cells (for e.g., as in WO2019126634, hereby incorporated by reference).

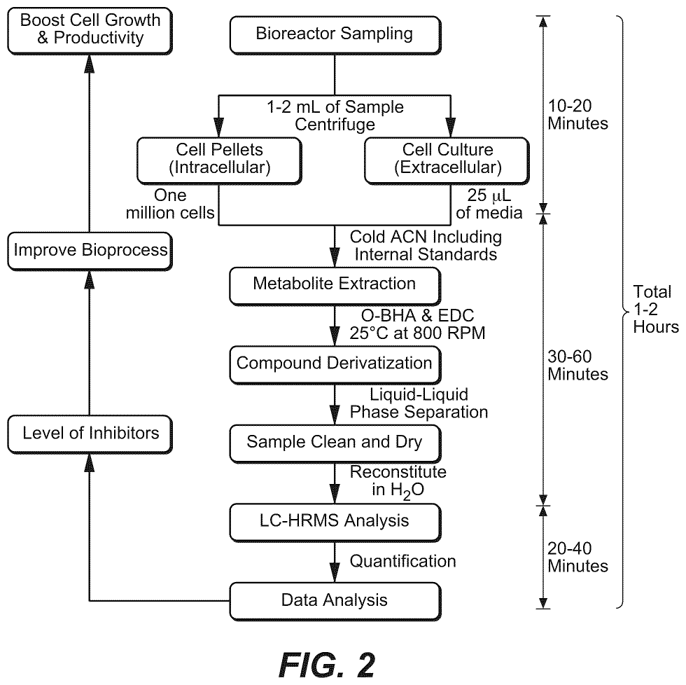

The method may further comprise on-line sampling of the cell culture to obtain the sample. The on-line sampling may comprise use of a commercially available on-line sampling system that may be adapted by the skilled person for use in the methods of present disclosure and invention. Exemplary on-line sampling systems include SegFlow from Flownamics, MAST from Lonza/Bend Research, and Numera from SecureCell. The on-line sampling may comprise pumping a volume of solution (e.g. a volume of from about 1 mL to about 20 mL) from the cell culture. Where the method comprises on-line sampling of the cell culture, said quantifying may be completed within about 8 hours, within about 6 hours, within about 4 hours, within about 2 hours, or within about 1 hour of on-line sampling of the cell culture. In embodiments, said quantifying is completed within about 4 hours of on-line sampling of the cell culture. In embodiments, said quantifying is completed within about 2 hours of on-line sampling of the cell culture. Exemplary timescales are illustrated in FIG. 2. For example, steps (a) and (b) of methods in accordance with the first aspect of the invention may be completed within about 30 to about 40 minutes, and steps (c) and (d) of methods in accordance with the first aspect of the invention may be completed within about 20 to about 40 minutes, as indicated in FIG. 2.

Each internal standard may correspond to an isotopologue of at least two nominal mass units more than the most abundant isotopologue of one of the multiple analytes. For example, the isotopologue may be two, three, four, five or six nominal mass units more than the most abundant isotopologue of one of the multiple analytes. Preferably, the isotopologue is two, three or four nominal mass units more than the most abundant isotopologue of one of the multiple analytes.

Each internal standard may comprise an isotopologue of one of the multiple metabolites having a double, triple, quadruple, a quintuple, or a setuple isotopic label, optionally wherein each isotopic label is selected from a deuterium, a carbon-13 (¹³C), or nitrogen-15 (¹⁵N). Preferably, each internal standard may comprise an isotopologue of one of the multiple metabolites having a double, triple, or quadruple, isotopic label, optionally wherein each isotopic label is selected from a deuterium, a carbon-13 (¹³C), or nitrogen-15 (¹⁵N).

Each internal standard may correspond to a doubly, triply, quadruply, quintuply, or sextuply deuterated isotopologue of one of the multiple analytes. Preferably, each internal standard corresponds to a doubly, triply, or quadruply deuterated isotopologue of one of the multiple analytes.

The one or more multiple metabolites may be inhibitory metabolites. The multiple metabolites may comprise or consist of one or more amino acid derived metabolites.

In embodiments, the multiple metabolites comprise or consist of one or more of formic acid, butyric acid, isobutyric acid, isovaleric acid, caproic acid, 2-methylbutyric acid, 2-hydroxybutryic acid, 3-hydroxybutyric acid, 2-hydroxyisovaleric acid, 2-hydroxyisocaproic acid, α-ketoisovaleric acid, α-ketoisocaprioc acid, indole-3-acetic acid, indole-3-lactic acid, indole-3-propionic acid, phenylacetic acid, phenyllactic acid, phenylpyruvic acid, 4-hydroxyphenylacetic acid, 4-hydroxyphenyllactic acid, 4-hydroxyphenylpyruvic acid, valine, leucine, isoleucine, aspartic acid, tryptophan, malate, fumarate, succinate, α-ketoglutaric acid.

In embodiments, the multiple metabolites comprise or consist of one or more of formic acid, butyric acid, isobutyric acid, isovaleric acid, caproic acid, 2-methylbutyric acid, 2-hydroxybutryic acid, 3-hydroxybutyric acid, 2-hydroxyisovaleric acid, 2-hydroxyisocaproic acid, α-ketoisovaleric acid, α-ketoisocaprioc acid, indole-3-acetic acid, indole-3-lactic acid, indole-3-propionic acid, phenylacetic acid, phenyllactic acid, phenylpyruvic acid, 4-hydroxyphenylacetic acid, 4-hydroxyphenyllactic acid, and 4-hydroxyphenylpyruvic acid.

In embodiments, the internal standard corresponding to each of the multiple metabolites is: butyric acid-D2 when butyric acid is a said metabolite; isobutyric acid-D3 when isobutyric acid and/or formic acid are said metabolites; isovaleric acid-D2 when isovaleric acid and/or 2-methylbutyric acid are said metabolites; caproic acid-D2 when caproic acid is a said metabolite; 3-hydroxybutyric acid-D4 when 2-hydroxybutyric acid and/or 3-hydroxybutyric acid and/or 2-hydroxyisovaleric acid and/or 2-hydroxyisocaproic acid are said metabolites; α-ketoisocaproic acid-D3 when α-ketoisovaleric acid and/or α-ketoisocaproic acid are said metabolites; indole-3-acetic acid-D2 when indole-3-acetic acid and/or indole-3-lactic acid and/or indole-3-propionic acid are said metabolites; phenyllactic acid-D3 when phenylacetic acid and/or phenyllactic acid and/or phenylpyruvic acid are said metabolites; and 4-hydroxyphenyllactic acid-D3 when 4-hydroxyphenylacetic acid and/or 4-hydroxyphenyllactic acid and/or 4-hydroxyphenylpyruvic acid are said metabolites.

Step (b) comprises derivatising the carbonyl group with a reagent comprising a carbonyl (e.g. a carboxyl) reactive group and a masking group. As the skilled person will be aware, there are different reagents and reaction chemistries that may be used to provide such a derivatisation and any suitable reagents may be used. Suitable reagents may comprise a masking group that is suitable for mass spectrometric detection. A number of exemplary derivatising reagents for carboxylic acids are indicated in Table 1. Table 2 indicates other exemplary derivatising reagents for carboxylic acids that may be used prior to gas chromatography (GO) chromatographic separation.

TABLE 1
Exemplary carboxylic acid derivatising reagents

| Carboxylic acid Derivatization Reagent | Reference |
|---|---|
| Hydrazinopyridine (HP) | J Pharm B [symbol] o [symbol] ed Anal [symbol] 2010 |
| | Sep. 5 [symbol] 52(5) [symbol] 809-18. do [symbol] |
| | 10 [symbol] 1016/ [symbol] ba.2010.03.001. |
| 2-picolylamine (PA) | J Pharm B [symbol] o [symbol] ed Anal [symbol] 2010 |
| | Sep. 5 [symbol] 52(5) [symbol] 809-18. doi [symbol] |
| | 10 [symbol] 1016/ [symbol] ba.2010.03.001 |
| 4-bromo-N-methylbenzylamine (4-BNMA) | Chromatographia. |
| | 2017 [symbol] 80(12) [symbol] 1723-1732 [symbol] doi [symbol] |
| | 10 [symbol] 1007/s10337-017-3421-0 [symbol] |
| | Epub 2017 Oct. 29. |
| 3-methyl-N-methylbenzylamine (3-BNMA) | Chromatographia. |
| | 2017 [symbol] 80(12) [symbol] 1723-1732 [symbol] doi [symbol] |
| | 10 [symbol] 1007/s10337-017-3421-0 [symbol] |
| | Epub 2017 Oct. 29. |
| 4-bromo-n-methylaniline (4-BMA) | Chromatographia. |
| | 2017 [symbol] 80(12) [symbol] 1723-1732 [symbol] doi [symbol] |
| | 10 [symbol] 1007/s10337-017-3421-0 [symbol] |
| | Epub 2017 Oct. 29. |
| 2-(4-Bromophenyl)-N-methylethanamine (B-MPEA) | Chromatographia. |
| | 2017 [symbol] 80(12) [symbol] 1723-1732 [symbol] doi [symbol] |
| | 10 [symbol] 1007/s10337-017-3421-0 [symbol] |
| | Epub 2017 Oct. 29 |
| 2-Hydrazinoquinoline (HQ) | Metabolites [symbol] 2013 Oct. |
| | 31 [symbol] 3(4) [symbol] 993-1010 [symbol] doi [symbol] |
| | 10 [symbol] 3390/metabo3040993 [symbol] |
| dimethylaminophenacyl bromide (DmPABr) | J Chromatogr A [symbol] 2019 Dec. |
| | 20 [symbol] 1608 [symbol] 460413 [symbol] doi [symbol] |
| | 10 [symbol] 1016/j.chroma.2019.460413. |
| 4-(2-(trimethylammonio)ethoxy)benzeneaminium | Anal Bioanal Chem. 2010 |
| dibromide (4-APC) | May [symbol] 397(2) [symbol] 665-75 [symbol] doi [symbol] |
| | 10 [symbol] 1007/s00216-010-3575-1. |
| -(2-((4- | Anal Bioanal Chem. 2010 |
| bromophenethyl)dimethylammonio)ethoxy)benzenaminium | May [symbol] 397(2) [symbol] 665-75 [symbol] doi [symbol] |
| dibromide (4-APEBA) | 10 [symbol] 1007/s00216-010-3575-1. |
| N-(4-aminomethylphenyl)pyridinium (AMPP) | Anal Chem. 2010 Aug. |
| | 15 [symbol] 82(16) [symbol] 6790-6 [symbol] doi [symbol] |
| | 10 [symbol] 1021/ac100720 [symbol] . |

[symbol]

indicates data missing or illegible when filed

TABLE 2
Exemplary carboxylic acid derivatising reagents for use prior to GC separation

| Derivatization reagents or reactions of | |
|---|---|
| carboxylic acids for GC MS analysis | Reference |
| 4-t-Butylbenzylation | SN Applied Sciences volume 2 [symbol] Article |
| | number [symbol] 856 (2020) |
| Methyl chloroformate (MCF) | Anal Bioanal Chem. 2016 |
| | Mar [symbol] 408(8) [symbol] 2171-83 [symbol] doi [symbol] 10 [symbol] 1007/s00216- |
| | 016-9321-6. |
| 2,4-difluoroaniline (DFA) | [symbol] nt J Environ Res Public Health. 2020 Jan [symbol] |
| | 17(1) [symbol] 100. |
| 10% BF3 (Boron trifluoride) in 1-butanol | Talanta [symbol] 2012 May 15 [symbol] 93 [symbol] 224-32 [symbol] doi [symbol] |
| | 10 [symbol] 1016/ [symbol] talanta.2012.02.022. |
| Trimethylanilium hydroxide (TMPAH) | Talanta [symbol] 2012 May 15 [symbol] 93 [symbol] 224-32 [symbol] doi [symbol] |
| | 10 [symbol] 1016/ [symbol] talanta.2012.02.022. |
| 1% trimethylsilyl chloride (TMCS) in | Talanta [symbol] 2012 May 15 [symbol] 93 [symbol] 224-32 [symbol] doi [symbol] |
| N,O- | 10 [symbol] 1016/ [symbol] talanta.2012.02.022. |
| Bis(trimethylsilyl)trifluoroacetamide | |
| (BSTFA) | |
| Trimethylsilylation | TrAC Trends in Analytical Chemistry |
| N-Methyl-N-(trimethylsilyl) trifluoroacetamide | Volume 61 [symbol] October 2014 [symbol] Pages 17-28 |
| (MSTFA) | |
| Bis(trimethylsilyl)acetamide (BSA) | |
| N,O-Bis(trimethylsilyl) trifluoroacetamide | |
| (BSTFA) | |
| Trimethylchlorosilane (TMCS) | |
| t-butyldimethylsilylation | TrAC Trends in Analytical Chemistry |
| N-methyl-N-tert- butyldimethylsilyl | Volume 61 [symbol] October 2014 [symbol] Pages 17-28 |
| trifluoroacetamide (MtBSTFA) | |
| Methyl ester formation | TrAC Trends in Analytical Chemistry |
| Trimethylsulfonium hydroxide (TMSH) | Volume 61 [symbol] October 2014 [symbol] Pages 17-28 |
| Dimethyl sulfate (DMS) | |
| Methanol/inorganic acid | |
| Methanol/Acyl chloride | |
| Methanol/Boron trifluoride (BF₃) | |
| Esterification | TrAC Trends in Analytical Chemistry |
| Alcohol/inorganic acid Alcohol/BF₃ | Volume 61 [symbol] October 2014 [symbol] Pages 17-28 |
| Pentafluorobenzyl bromide (PFBBr) | |
| Picolinyl esters (3-Pyridylcarbinol esters) | |
| Dimethyloxazoline formation | TrAC Trends in Analytical Chemistry |
| 4,4-dimethyloxazoline (DMOX) | Volume 61 [symbol] October 2014 [symbol] Pages 17-28 |

[symbol]

indicates data missing or illegible when filed

The step (b) of contacting may further comprise activating the carbonyl group of each metabolite, thereby providing an activated carbonyl group on each metabolite for reaction with the carbonyl or carboxyl reactive group. Activating the carbonyl group of each metabolite may comprise contacting the sample with N-(3-dimethylaminopropyl)-N′-ethylcarbodiimide (EDC) or a salt thereof, such as EDC hydrochloride.

The carbonyl reactive group of the reagent comprising the carbonyl reactive group and the masking group may comprise an amine. The method of any preceding claim, wherein the masking group of the reagent comprising the carbonyl reactive group and the masking group comprises an aliphatic group and/or an aromatic group. For example, the reagent comprising the carbonyl reactive group and the masking group may be O-benzylhydroxylamine (O-BHA), or a salt thereof, such as O-BHA hydrochloride.

The chromatographic separation may comprise reverse phase liquid chromatography, hydrophilic interaction liquid chromatography (HILIC), or gas chromatography (GC). The chromatographic separation may comprise reverse phase liquid chromatography or hydrophilic interaction liquid chromatography (HILIC). In embodiments, the chromatographic separation comprises reverse phase liquid chromatography. The reverse phase liquid chromatography may comprise gradient elution from a C18, C8 or phenyl-hexyl column, preferably a C18 column. The chromatographic separation may comprise HILIC. The chromatographic separation may comprise GC.

The mass spectrometry may comprise introducing the eluate from the chromatographic separation into a mass spectrometer ion source to generate positive ions of analyte molecules and performing and obtaining full scan mass spectra on an accurate mass high resolution mass spectrometer.

The positive ions of analyte molecules may comprise a cation selected from a proton, a sodium ion, a potassium ion, a lithium ion, or an ammonium ion; for example a proton, a sodium ion, a potassium ion, or an ammonium ion. In embodiments, the positive ions are protonated molecules.

Any mass spectrometer that is compatible with liquid samples comprising cell culture media and/or cell lysates may be used in exemplary methods of the invention. The mass spectrometer may comprise an atmospheric pressure ionization source, such as electrospray ionization (ESI), atmospheric pressure chemical ionization (APCI), or atmospheric pressure photo-ionization (APPI); for example the ionization source may be ESI. Where the chromatographic separation comprises GC, any mass spectrometer that is compatible with gas samples may be used in the methods; for example the ionization source may be EI, C, or the like.

The mass spectrometer may comprise an analyzer comprising an Orbitrap, a quadrupole ion trap, a linear ion trap, a time-of-flight, a quadrupole, a triple quadrupole, Fourier-transform ion cyclotron resonance (FTICR), or a combination thereof (such as a quadrupole and/or an ion trap combined with an Orbitrap, time-of-flight, or FTICR analyzer). The mass spectrometer may be a high resolution and high mass accuracy mass spectrometer. For example, the mass spectrometer may comprise an Orbitrap or FTICR mass analyzer, e.g. the mass spectrometer may comprise an Orbitrap mass analyzer. For example, the mass spectrometer may be a Q-Extractive Orbitrap mass spectrometer.

The full scan accurate mass high resolution mass spectrometry may comprise scanning a mass to charge ratio of from at least about 50 to not more than about 800 m/z. In embodiments, the full scan accurate mass high resolution mass spectrometry comprises scanning a mass to charge ratio of from about 80 to about 600 m/z. In embodiments, the full scan accurate mass high resolution mass spectrometry comprises scanning a mass to charge ratio of from about 100 to about 500 m/z.

The mass accuracy of the mass spectrometry may be 20 ppm or better, may be 10 ppm or better, or may be 5 ppm or better. In embodiments, the mass accuracy of the mass spectrometry is about 5 ppm.

The resolution of the mass spectrometry may be at least 35,000, at least 70,000, or at least 140,000. In embodiments, the resolution of the mass spectrometry is about 70,000. The resolution may be based upon m/Δm using the full width of the peak at half its maximum height (FWHM) definition, where m is the mass of the ion and Δm is the width of the peak at half of its height.

The dynamic range for quantifying the concentration of each of the multiple metabolites may be at least 2.5 orders of magnitude, at least 3 orders of magnitude or at least 3.5 orders of magnitude. In embodiments, the concentration of each of the multiple metabolites is at least 3 orders of magnitude.

### Methods of Relative Quantification of Multiple Metabolites

Provided herein are methods for the relative quantification of multiple metabolites in a sample. These methods provide the same advantages as discussed for the above methods of absolute quantification of the concentration of multiple metabolites. For example, the present methods allow rapid determination of metabolite levels. This may allow close to real-time (e.g. within 2 or so hours) determination of metabolite levels in a cell culture, allowing for timely adjustment of cell culture conditions in response to metabolite levels. This assists in optimising cell culture conditions and may assist in maximising product quantity and/or quality. For example, productivity and/or titre may be improved. In addition, methods of relative quantification do not involve the presence of internal standard in the cell culture sample. This is particularly advantageous where it is not known which metabolite(s) in a sample will have the desired inhibitory effect before performing the method. Additionally, the reduced number of components in the metabolite sample simplifies purification of the sample.

Relative quantification may, for example, be particularly useful where the relative concentrations of certain metabolites correlate well with culture growth and performance. Such data, along with use of proper algorithms and machine learning may be used to predict culture growth and behaviour during, e.g., N−1 and N phases of production.

An embodiment provides a method for relative quantification of multiple metabolites in a sample, wherein each metabolite comprises at least one carbonyl group, the method comprising: (a) contacting the sample with a reagent comprising a carbonyl reactive group and a masking group, thereby derivatising the carbonyl group of each metabolite with the masking group and providing a derivatised sample; (b) subjecting the derivatised sample to chromatographic separation and full scan accurate mass high resolution mass spectrometry; and (c) quantifying the amount of each of the multiple metabolites based on a monoisotopic signal obtained for each said metabolite, wherein said quantifying comprises comparing the monoisotopic signal obtained for each said one of the multiple metabolites to the monoisotopic signal obtained for each said other of the multiple metabolites, thereby obtaining the relative quantification,
wherein the monoisotopic signal obtained for each one of the metabolites corresponds to the most abundant positive ion isotopologue, unless said most abundant positive ion isotopologue has a signal at or above the upper limit of quantification for said metabolite, in which case the monoisotopic signal obtained for said metabolite corresponds to a less abundant positive ion isotopologue of said metabolite.

The at least one carbonyl group may be selected from a carboxylic acid, a ketone and an aldehyde. The at least one carbonyl group may be a carboxylic acid or a ketone. The at least one carbonyl group may be a carboxylic acid. The at least one carbonyl group may be a ketone.

The carbonyl reactive group may be selected from a carboxyl acid reactive group, a ketone reactive group and an aldehyde reactive group. The carbonyl reactive group may be a carboxylic acid reactive group or a ketone reactive group. The carbonyl reactive group may be a carboxylic acid reactive group. The carbonyl reactive group may be a ketone reactive group.

Preferably, the at least one carbonyl group is a carboxylic acid and the carbonyl reactive group is a carboxylic acid reactive group. Alternatively, the at least one carbonyl group is a ketone and the carbonyl reactive group is a ketone reactive group.

The sample may be a sample of a cell culture. For example, the sample may be a cell culture medium and/or the sample may be a cell culture medium of cell lysate. In embodiments, the sample is a cell culture medium. The cell culture may be an animal cell culture, e.g. a mammalian cell culture or an insect cell culture. Alternatively, the cell culture may be a fungal cell culture, e.g. a yeast cell culture.

The cell culture may be a hybridoma culture, CHO cell culture, COS cell culture, VERO cell culture, HeLa cell culture, HEK 293 cell culture, PER-C6 cell culture, K562 cell culture, MOLT-4 cell culture, MI cell culture, NS-1 cell culture, COS-7 cell culture, MDBK cell culture, MDCK cell culture, MRC-5 cell culture, WI-38 cell culture, WEHI cell culture, SP2/0 cell culture, or BHK cell culture (including BHK-21 cell), or any derivative thereof.

The cell culture may be a CHO cell culture. In embodiments, the CHO cell culture is a CHO K1 cell culture, a CHO K1SV cell culture, a DG44 cell culture, a DUKXB-11 cell culture, a CHOK1S cell culture, or a CHO K1M cell culture, or a derivative thereof. Derivatives of said CHO cell culture may comprise CHO K1, CHO K1SV, DG44, DUKXB-11, CHOK1S, or any CHO cell or CHO derivative cell engineered for targeted integration of a gene of interest from said cells (for e.g., as in WO2019126634, hereby incorporated by reference).

The method may further comprise on-line sampling of the cell culture to obtain the sample. The on-line sampling may comprise use of a commercially available on-line sampling system that may be adapted by the skilled person for use in the methods of present disclosure and invention. Exemplary on-line sampling systems include SegFlow from Flownamics, MAST from Lonza/Bend Research, and Numera from SecureCell. The on-line sampling may comprise pumping a volume of solution (e.g. a volume of from about 1 mL to about 20 mL) from the cell culture. Where the method comprises on-line sampling of the cell culture, said quantifying may be completed within about 8 hours, within about 6 hours, within about 4 hours, within about 2 hours, or within about 1 hour of on-line sampling of the cell culture. In embodiments, said quantifying is completed within about 4 hours of on-line sampling of the cell culture. In embodiments, said quantifying is completed within about 2 hours of on-line sampling of the cell culture. The ability to provide quantification of multiple metabolites within a few hours provides advantages, for example it may allow the skilled person to stop and/or optimise the conditions in the cell culture before any said metabolite has reached an undesirable level.

The one or more multiple metabolites may be inhibitory metabolites. The multiple metabolites may comprise or consist of one or more amino acid derived metabolites.

In embodiments, the multiple metabolites comprise or consist of one or more of formic acid, butyric acid, isobutyric acid, isovaleric acid, caproic acid, 2-methylbutyric acid, 2-hydroxybutryic acid, 3-hydroxybutyric acid, 2-hydroxyisovaleric acid, 2-hydroxyisocaproic acid, α-ketoisovaleric acid, α-ketoisocaprioc acid, indole-3-acetic acid, indole-3-lactic acid, indole-3-propionic acid, phenylacetic acid, phenyllactic acid, phenylpyruvic acid, 4-hydroxyphenylacetic acid, 4-hydroxyphenyllactic acid, 4-hydroxyphenylpyruvic acid, valine, leucine, isoleucine, aspartic acid, tryptophan, malate, fumarate, succinate, α-ketoglutaric acid.

In embodiments, the multiple metabolites comprise or consist of one or more of formic acid, butyric acid, isobutyric acid, isovaleric acid, caproic acid, 2-methylbutyric acid, 2-hydroxybutryic acid, 3-hydroxybutyric acid, 2-hydroxyisovaleric acid, 2-hydroxyisocaproic acid, α-ketoisovaleric acid, α-ketoisocaprioc acid, indole-3-acetic acid, indole-3-lactic acid, indole-3-propionic acid, phenylacetic acid, phenyllactic acid, phenylpyruvic acid, 4-hydroxyphenylacetic acid, 4-hydroxyphenyllactic acid, and 4-hydroxyphenylpyruvic acid.

Step (b) comprises derivatising the carbonyl group with a reagent comprising a carbonyl reactive group and a masking group. As the skilled person will be aware, there are different reagents and reaction chemistries that may be used to provide such a derivatisation and any suitable reagents may be used. Suitable reagents may comprise a masking group that is suitable for mass spectrometric detection. A number of exemplary derivatising reagents for carboxylic acids are indicated in Table 1 above. Table 2 above indicates other exemplary derivatising reagents for carboxylic acids that may be used prior to gas chromatography (GC) chromatographic separation.

The step (b) of contacting may further comprise activating the carbonyl group of each metabolite, thereby providing an activated carbonyl group on each metabolite for reaction with the carbonyl reactive group. Activating the carbonyl group group of each metabolite may comprise contacting the sample with N-(3-dimethylaminopropyl)-N′-ethylcarbodiimide (EDC) or a salt thereof, such as EDC hydrochloride.

The carbonyl reactive group of the reagent comprising the carbonyl reactive group and the masking group may comprise an amine. The method of any preceding claim, wherein the masking group of the reagent comprising the carbonyl reactive group and the masking group comprises an aliphatic group and/or an aromatic group. For example, the reagent comprising the carbonyl reactive group and the masking group may be O-benzylhydroxylamine (O-BHA), or a salt thereof, such as O-BHA hydrochloride.

The chromatographic separation may comprise reverse phase liquid chromatography, hydrophilic interaction liquid chromatography (HILIC), or gas chromatography (GC). The chromatographic separation may comprise reverse phase liquid chromatography or hydrophilic interaction liquid chromatography (HILIC). In embodiments, the chromatographic separation comprises reverse phase liquid chromatography. The reverse phase liquid chromatography may comprise gradient elution from a C18, C8 or phenyl-hexyl column, preferably a C18 column. The chromatographic separation may comprise HILIC. The chromatographic separation may comprise GC. The mass spectrometry may comprise introducing the eluate from the chromatographic separation into a mass spectrometer ion source to generate positive ions of analyte molecules and performing and obtaining full scan mass spectra on an accurate mass high resolution mass spectrometer.

The positive ions of analyte molecules may comprise a cation selected from a proton, a sodium ion, a potassium ion, a lithium ion, or an ammonium ion; for example a proton, a sodium ion, a potassium ion, or an ammonium ion. In embodiments, the positive ions are protonated molecules.

Any mass spectrometer that is compatible with liquid samples comprising cell culture media and/or cell lysates may be used in the methods of the invention. The mass spectrometer may comprise an atmospheric pressure ionization source, such as electrospray ionization (ESI), atmospheric pressure chemical ionization (APCI), or atmospheric pressure photo-ionization (APPI); for example the ionization source may be ESI. Where the chromatographic separation comprises GC, any mass spectrometer that is compatible with gas samples may be used in the methods. For example, the ionization source may be EI, CI, or the like.

The mass spectrometer may comprise an analyzer comprising an Orbitrap, a quadrupole ion trap, a linear ion trap, a time-of-flight, a quadrupole, a triple quadrupole, Fourier-transform ion cyclotron resonance (FTICR), or a combination thereof (such as a quadrupole and/or an ion trap combined with an Orbitrap, time-of-flight, or FTICR analyzer). The mass spectrometer may be a high resolution and high mass accuracy mass spectrometer. For example, the mass spectrometer may comprise an Orbitrap or FTICR mass analyzer, e.g. the mass spectrometer may comprise an Orbitrap mass analyzer. For example, the mass spectrometer may be a Q-Extractive Orbitrap mass spectrometer.

The full scan accurate mass high resolution mass spectrometry may comprise scanning a mass to charge ratio of from at least about 50 to not more than about 800 m/z, In embodiments, the full scan accurate mass high resolution mass spectrometry comprises scanning a mass to charge ratio of from about 80 to about 600 m/z. In embodiments, the full scan accurate mass high resolution mass spectrometry comprises scanning a mass to charge ratio of from about 100 to about 500 m/z.

The mass accuracy of the mass spectrometry may be 20 ppm or better, may be 10 ppm or better, or may be 5 ppm or better. In embodiments, the mass accuracy of the mass spectrometry is about 5 ppm.

The resolution of the mass spectrometry may be at least 35,000, at least 70,000, or at least 140,000. In embodiments, the resolution of the mass spectrometry is about 70,000. The resolution may be based upon m/Δm using the full width of the peak at half its maximum height (FWHM) definition, where m is the mass of the ion and Δm is the width of the peak at half of its height.

The dynamic range for quantifying the concentration of each of the multiple metabolites may be at least 2.5 orders of magnitude, at least 3 orders of magnitude or at least 3.5 orders of magnitude. In embodiments, the concentration of each of the multiple metabolites is at least 3 orders of magnitude.

### Methods of Monitoring the Course of a Cell Culture

An embodiment provides a method of monitoring the course of a cell culture, comprising:
(a) performing a method of the first or second aspect at a first time point, wherein the sample is a sample of a cell culture;
(b) repeating the method of said first or second aspect at a second time point; and
(c) optionally repeating the method of said first or second aspect at one or more subsequent time points,
wherein the monitoring comprises comparing the levels of each of the multiple metabolites between each of the multiple time points

The monitoring may further comprise multivariate statistical analysis of the levels of each of the multiple metabolites, for example to determine correlation between metabolite levels and cell culture properties.

The cell culture property may be selected from cell growth, cell viability, product titre, product quality, productivity and the like.

### Methods of Optimising a Cell Culture

An embodiment provides a method of optimising a cell culture, comprising: performing a method of quantifying the concentration of multiple metabolites in a sample at multiple time points, and, in response to a change in the concentration of at least one of the multiple metabolites characterized at a later timepoint compared to an earlier timepoint, adjusting a cell culture condition.

Without wishing to be bound by any theory, we have determined that, when inhibitory metabolites go above a certain level, the inhibitory effect may remain for at least a period of time, even after cell culture is refreshed. The methods of optimising cell culture of the disclosure therefore provide advantages, as the rapid analysis of multiple metabolites provided by the present methods allows optimising actions (e.g. perfusion or dilution to reduce inhibitory metabolite levels) to be taken in a timely manner.

The cell culture may, for example, be a batch culture, a fed batch culture, or a perfusion culture. Performing the method at more than one time point during the course of a cell culture (or comparing to results from a previous or parallel cell culture) will reveal whether the monitored sample attributes are within specification, or if they are trending or out of specification. Where a sample attribute is trending or out of specification, the cell culture conditions are adjusted to bring the attribute(s) back towards the specification setpoint. Alternatively, the cell culture could be optimized by halting the process and isolating the monitored sample when the attributing is trending away from the specification setpoint, but is still within the specification.

In such methods of optimizing cell culture, the ability to measure the sample attribute with high accuracy is critical and is provided by the methods described herein. Furthermore, the rapid timescale of the measurement (potentially every 2 hours) is advantageous, as it may permit relatively tight control on the attribute of interest.

The change in concentration may comprise an increase in concentration of at least one of the multiple metabolites between the first time point and a second or subsequent time point. The said at least one of the multiple metabolites may be an inhibitor.

The cell culture condition may be selected from perfusion rate, temperature, pH, dissolved oxygen (dO₂), cell culture duration, and the level of one or more cell culture ingredient(s) such as amino acids, vitamins, inorganic salts, sugars (e.g. glucose), buffering salts and lipids. The cell culture condition may be or may comprise perfusion rate. The cell culture condition may be or may comprise temperature. The cell culture condition may be or may comprise pH. The cell culture condition may be or may comprise dO₂. The cell culture condition may be or may comprise cell culture duration, for example wherein adjusting the cell culture condition comprises stopping the cell culture. The cell culture condition may be or may comprise the level of one or more cell culture ingredients, for example, one or more of amino acids, vitamins, inorganic salts, sugars (e.g. glucose), buffering salts, lipids, trace elements, and small molecules to improve growth or productivity; e.g. one or more of amino acids, vitamins, inorganic salts, sugars (e.g. glucose), buffering salts and lipids.

### Examples

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Materials and Methods

The following reagents and authentic standard compounds were obtained from the named suppliers: water, methanol and acetonitrile were ordered as LC-MS grade from Fisher Scientific (Waltham, MA, USA); dichloromethane (DCM), O-benzylhydroxylamine hydrochloride (O-BHA) and N-(3-dimethylaminopropyl)-N′-ethylcarbodiimide hydrochloride (EDC) were ordered from Sigma Aldrich (St. Louis, MO, USA); formic acid, butyric acid, isobutyric acid, isovaleric acid, caproic acid, 2-methylbutyric acid, 2-hydroxybutyric acid, 3-hydroxybutyric acid, 2-hydroxyisovaleric acid, 2-hydroxyisocaproic acid, alpha-ketoisovaleric acid, alpha-ketoisocaproic acid, indole-3-acetic acid, indole-3-lactic acid, indole-3-propionic acid, phenylacetic acid, phenyllactic acid, phenylpyruvic acid, 4-hydroxyphenylacetic acid, 4-hydroxyphenyllactic acid, and 4-hydroxyphenylpyruvic acid were purchased from Sigma Aldrich (St. Louis, MO, USA); butyric acid-D2, isobutyric acid-D3, isovaleric acid-D2, and caproic acid-D2 were obtained from CDN Isotopes (Pointe-Claire, QC, Canada); 3-hydroxybutyric acid-D4 and phenyllactic acid-D3 were obtained from Toronto Research Chemicals (Toronto, ON, Canada); alpha-ketoisocaproic acid-D3 were obtained from Cambridge Isotope Laboratories (Tewksbury, MA, USA); indole-3-acetic acid-D2 were obtained from Sigma Aldrich (St. Louis, MO, USA); 4-hydroxyphenyllactic acid-D3 were obtained from Medical Isotopes (Pelham, NH, USA).

An exemplary method of the invention is depicted in FIG. 2. Examples 1, 2 and 3 below describe this method in further detail.

### Example 1: Cell Culture

High seeding density fed-batch production cultures were performed in shake flasks with proprietary chemically defined production media. In cell that are intended for use in biotechnological production, e.g. of protein products such as antibodies, may be cultured through multiple stages of bioreactors. In such culturing processes, the N stage bioreactor refers to the production bioreactor, while the N−1 stage refers to the bioreactor in the inoculum train immediately preceding the N stage bioreactor (note that N−1 reads “N minus 1” referring to the CHO culture before the N stage that is used to set up the N culture).

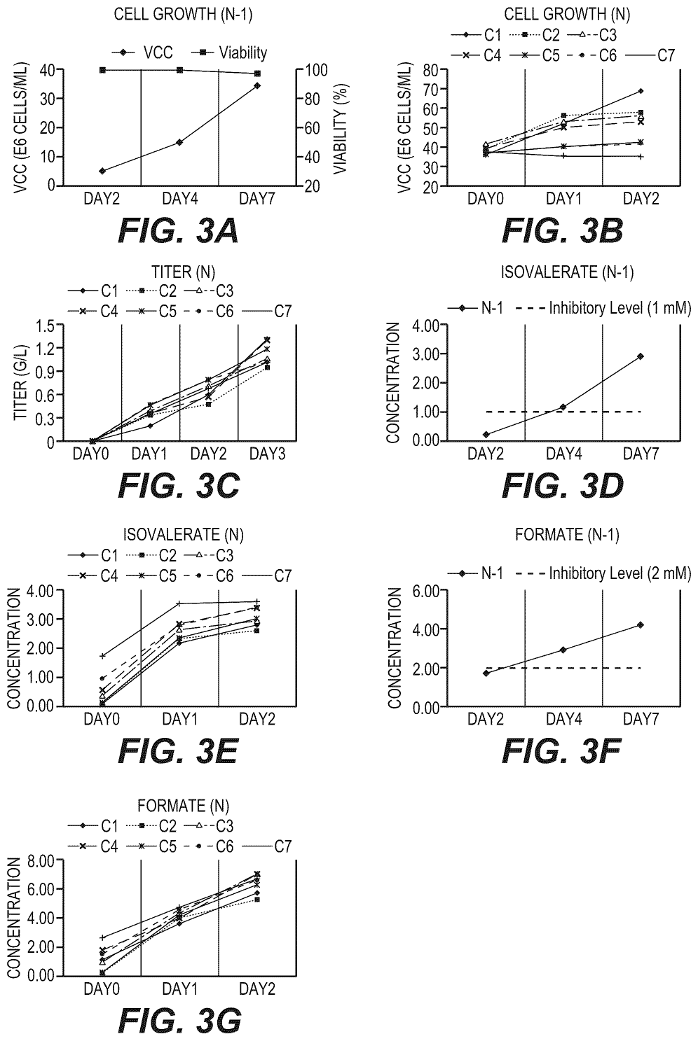

For the N−1 inoculum train, cells were seeded at 2×10⁶ cells/ml on Day 0 and maintained for 7 days with feed media and glucose addition on day 4. On day 2, 4 and 7 of the N−1 culture, inoculum train cells were seeded into the production cultures by either complete or partial media exchange (1:1 or 3:1 fresh media to spent media ratio) to potentially mimic fully or partially perfused cultures, setting up production culture with 3×10⁷ cells/mL starting cell density on Day 0. The short 3 days production run was set up to illustrate the effects of inhibitory molecules on cell growth obtained from N−1 cultures, where over time cells were exposed to increasingly higher concentrations of the accumulated inhibitory molecules. The intensified shake flask production process mimics the intensified N process in bioreactors, while the complete or partial media exchange used for setting up the production culture was used to mimic different perfusion speed in N−1. The results of this are also depicted in FIG. 3.

For sample collection, cell culture samples were acquired at interested time points. Collected samples were centrifuged to separate the cell pellets and supernatants. CHO cell fed-batch culture supernatants were transferred into clean tubes and submitted for further metabolomics analysis. Metabolites of interest were extracted from fed-batch culture samples using cold acetonitrile containing stable isotope-labeled internal standards mixture; and then derivatized with 10 μL of 0.3 M O-BHA (in MeOH) and 10 μL of 0.3 M EDC (in MeOH) at 800 RPM and 25° C. for 10 minutes. Subsequently, 50 μL of water and 400 μL of dichloromethane were added to perform liquid-liquid phase separation. Samples were mixed and centrifuged again. Organic layer aliquots containing derivatized compounds were transferred, dried, reconstituted in 100 μL of water, and submitted for further LC-HRMS analysis. Metabolomics data for targeted quantification of growth inhibition related metabolites in CHO cell fed-batch cultures were acquired and analyzed to obtain the absolute concentration results.

CHO cell N−1 inoculum cultures were used to set up production cultures at different days (Table 3). Fresh media was used to replace or dilute the spent N−1 culture media at the ratios shown. This series of dilution media represents the potential outcome that one might get from the perfusion process.

TABLE 3

| Condition | C1 | C2 | C3 | C4 | C5 | C6 | C7 |
|---|---|---|---|---|---|---|---|
| Inoculation | Day 2 | Day 4 | Day 4 | Day 4 | Day 7 | Day 7 | Day 7 |
| Day from N-1 | | | | | | | |
| Media | 1:1 | 0:1 | 1:3 | 1:1 | 0:1 | 1:3 | 1:1 |
| (Spent:Fresh) | | | | | | | |

### Example 2: Sampling, Extraction and Derivatisation

### Sample Collection

From each bioreactor, a 10 mL cell culture sample was acquired at interested time points. Collected samples were centrifuged to separate the cell pellets and supernatants. CHO cell fed-batch culture supernatants (and cell pellets if interested) were transferred into clean tubes and submitted for further metabolomics analysis.

### Metabolite Extraction

For metabolite extraction of cell culture supernatants (sample size−25 μL of media aliquot), 100 μL of cold acetonitrile containing stable isotope-labelled internal standards mixture (IS working solution) were added to each sample. Samples were vortexed and centrifuged at 4000 RPM for 5 minutes. And then 80 μL of sample supernatants were transferred to clean tubes for further derivatization.

For metabolite extraction of cell pellets (intracellular interests) (sample size−1 million cells), 250 μL of cold acetonitrile containing stable isotope-labeled internal standards mixture (IS working solution) were added to each sample. Samples were incubated at −80 freezer for 15 min, vortexed and centrifuged. And then 80 μL of sample supernatants were transferred to clean tubes for further derivatization.

### Compound Derivatization

For compound derivatization, 80 μL of sample supernatants were mixed with 10 μL of 0.3 M O-BHA (in MeOH) and 10 μL of 0.3 M EDC (in MeOH) at 800 RPM and 25° C. for 10 minutes. Subsequently, 50 μL of water and 400 μL of dichloromethane were added to perform liquid-liquid phase separation. Samples were mixed and centrifuged again. Organic layer aliquots containing derivatized metabolites of interest were transferred, dried, reconstituted in 100 μL of water, and submitted for further LC-HRMS analysis.

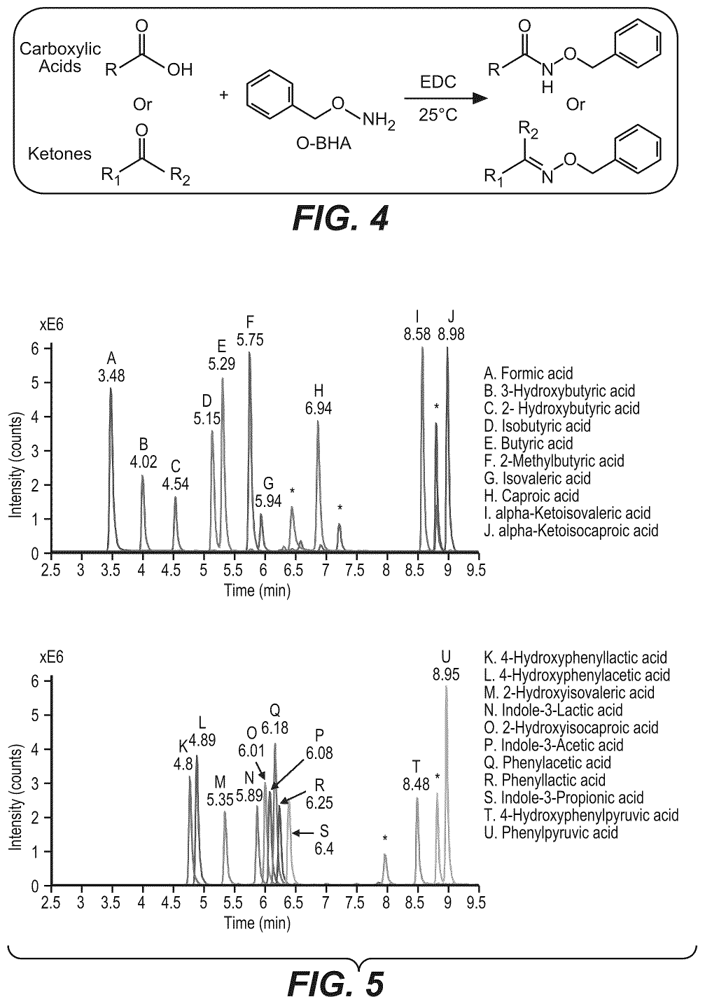

An exemplary derivatisation step is depicted in FIG. 4, in which carboxyl or ketone groups are activated with EDC at 25° C. and subjected to O-BHA derivatisation which results in the formation of amides or oximes, respectively. Performing this derivatization step in sample preparation procedures enhanced the chromatographic behavior and ionization efficiency of the target analytes, therefore contributing to an improvement in selectivity and sensitivity.

### Example 3: Chromatographic Separation and Analysis

### LC-HRMS Analysis

Metabolomics data for targeted quantification of growth inhibition related metabolites in CHO cell fed-batch cultures were acquired using Liquid Chromatography-High Resolution Mass Spectrometry (LC-HRMS) approach. Data collection was achieved on a Shimadzu Nexera HPLC series system (Shimadzu, Kyoto, Japan) coupled with a Thermo Q Exactive Plus Hybrid Quadrupole-Orbitrap Mass Spectrometer (Thermo Fisher Scientific, Waltham, MA, USA). Injection volume of 2 μL was used for sample analysis under Heated Electrospray Ionization (HESI) condition in positive ion mode. A Phenomenex Luna Omega C18 column (100 mm×2.1 mm×1.6 μm, 100 Å, Phenomenex, Torrance, CA, USA) was applied to chromatographically separate the analytes with mobile phase A of water containing 0.1% formic acid and mobile phase B of methanol only. The column temperature was maintained at 45° C. Total run time was 10.8 minutes with a flow rate of 0.5 mL/min. The LC gradient was 0 min, 10% B; 0.5 min, 10% B; 5 min, 55% B; 7.5 min, 65% B; 8 min, 85% B; 9.5 min, 85% B; 9.8 min, 10% B; 10.8 min, 10% B. The High-Resolution Q Exactive Plus mass spectrometer was operated with the following instrument parameters: sheath gas flow rate, 52 units; sweep gas flow rate, 3 units; aux gas flow rate, 14 units; aux gas temperature, 438° C.; capillary temperature, 269° C.; RF level for S-Lens, 40 units; spray voltage, 3500 V; scan mode, Full MS scan; scan range, 100-800 m/z; MS resolution, 70,000; AGC (Automatic Gain Control) target: 3xE6; maximum IT (Injection Time): 200 ms.

As demonstrated in FIG. 5, all metabolites of interest were well distributed based on either chromatographic separation or mass spectral resolution. For example, isomeric ion pairs, such as butyric acid and isobutyric acid, 2-hydroxybutyric acid and 3-hydroxybutyric acid, were effectively separated with different retention times (FIG. 5, upper LC-MS chromatogram); on the other hand, closely eluting compounds like 4-hydroxyphenylacetic acid and 4-hydroxyphenyllactic acid were completely resolved by accurate mass determination (FIG. 5, lower LC-MS chromatogram). This demonstrates the wide scope of metabolite screening achieved using the methods of the invention, as well as the validity and reliability of metabolite quantification.

### Data Analysis

Data acquisition software Xcalibur version 4.2 (Thermo Fisher Scientific, Waltham, MA, USA) and data processing software TraceFinder version 4.1 (Thermo Fisher Scientific, Waltham, MA, USA) were applied for LC-HRMS based targeted quantification. In TraceFinder, small molecule compound database for all 21 CHO cell growth inhibition related metabolites and 9 stable isotope-labeled internal standards (IS) including their retention times, derivatized chemical formulas, as well as accurate mass targeted ions, was constructed for compound identification. Then, peak detection and peak integration were achieved using closest RT strategy and high-resolution targeted ions with 5 ppm mass accuracy. Further calibration curves and quantification results were computed using linear regression model between analyte concentration and peak area ratio of analyte vs. corresponding IS. All analytes were assigned with specific internal standards based on their chromatographic or structural similarity. For quantification results with isotope analysis approach, response ratio was obtained through M+1 ion area of analyte vs. M ion area of corresponding internal standard. Next, concentration of naturally occurring M+1 isotope was determined by its response ratio compared to calibration curve of original M ion. The final concentration of analyte was derived using concentration of M+1 isotope divided by theoretical isotopic ratio of M+1/M. Microsoft Excel software was utilized for this series of calculation process.

Exemplary calibration curve information for both M ion-based quantification and M+1 ion-based quantification, including corresponding accurate mass target ions, curve slopes and coefficient R2 values are presented in Table 4. Concentration values for each of the calibration standards for all analytes are detailed in Table 5.

The matrix-matched calibration curve linearity was expressed through the Coefficient of Determination (R2) value of the linear regression model between analyte concentration and peak area ratio of analyte vs. corresponding internal standard. Calibration curve for quantification by protonated M target ion was computed using ten calibration standards (STD1-STD10); while calibration curve for quantification by protonated M+1 target ion was computed using eight calibration standards (STD3-STD10) due to ultra-low intensity of naturally occurring isotopes in STD1 and STD2. Matrix-matched calibration curve linearities were well validated with all R2 values >0.99 for M ion-based regression curves and >0.98 for M+1 ion-based regression curves.

The sensitivity of the assay was characterized through determination of the minimum concentration (LLOQ) of each target metabolite that could be quantified with acceptable accuracy and precision. The accuracy threshold was set as ±15%, indicating 85%-115% in absolute percentages, for all standard levels in the calibration curves, except ±20% for LLOQ. The LLOQ and ULOQ concentrations for target analytes based on regular M ion quantification are specified in Table 4. Widespread linear dynamic range across two orders of magnitude (100×) was achieved with LLOQ at 30-90 ng/mL and ULOQ at 3000-9000 ng/mL. Exception values for LLOQ at 1344 ng/mL and ULOQ at 80000 ng/mL were applied for formic acid owing to interference in sample processing.

TABLE 4

| | Calibration | | | Calibration | | | Isotopic Ratio | | | Limit of | |
|---|---|---|---|---|---|---|---|---|---|---|---|
| | Curve of M | | | Curve of M + 1 | | | of M + 1/M | | | Quantitation | |
| | Target | | | Target | | | Theo- | Experi- | Accu- | (ng/mL) | |
| Analyte | Ion | Slope | R2 | Ion | Slope | R2 | retical | mental | racy | LLOQ | ULOQ |
| Formic acid | 152.0706 | 8.4E−04 | 0.998 | 153.0740 | 7.2E−05 | 0.998 | 8.7% | 8.6% | 99.1% | 1344 | 80000 |
| Butyric acid | 194.1176 | 1.5E−02 | 0.999 | 195.1209 | 1.8E−03 | 0.994 | 11.9% | 11.8% | 99.0% | 50 | 5000 |
| Isobutyric acid | 194.1176 | 5.0E−03 | 0.999 | 195.1209 | 6.0E−04 | 0.994 | 11.9% | 11.9% | 99.7% | 50 | 5000 |
| Isovaleric acid | 208.1332 | 3.9E−03 | 0.998 | 209.1366 | 4.7E−04 | 0.999 | 13.0% | 12.2% | 93.6% | 50 | 5000 |
| Caproic acid | 222.1489 | 5.6E−03 | 0.998 | 223.1522 | 7.7E−04 | 0.997 | 14.1% | 13.9% | 98.7% | 90 | 9000 |
| 2-Methylbutyric acid | 208.1332 | 1.2E−02 | 0.999 | 209.1366 | 1.6E−03 | 0.999 | 13.0% | 12.5% | 96.4% | 50 | 5000 |
| 2-Hydroxybutyric acid | 210.1125 | 3.1E−03 | 0.997 | 211.1158 | 3.4E−04 | 0.995 | 11.9% | 10.9% | 91.8% | 50 | 5000 |
| 3-Hydroxybutyric acid | 210.1125 | 4.4E−03 | 0.999 | 211.1158 | 4.7E−04 | 0.998 | 11.9% | 10.7% | 90.2% | 50 | 5000 |
| 2-Hydroxyisovaleric acid | 224.1281 | 4.0E−03 | 0.996 | 225.1315 | 4.8E−04 | 0.996 | 13.0% | 11.8% | 91.1% | 50 | 5000 |
| 2-Hydroxyisocaproic acid | 238.1438 | 6.0E−03 | 0.997 | 239.1471 | 9.4E−04 | 0.992 | 14.1% | 15.6% | 111.2% | 50 | 5000 |
| alpha-Ketoisovaleric acid | 327.1703 | 2.9E−03 | 0.997 | 328.1737 | 5.8E−04 | 0.995 | 20.6% | 20.0% | 97.5% | 50 | 5000 |
| alpha-Ketoisocaproic acid | 341.1860 | 3.0E−03 | 0.999 | 342.1893 | 6.3E−04 | 0.998 | 21.6% | 21.0% | 97.1% | 50 | 5000 |
| Indole-3-Acetic acid | 281.1285 | 2.3E−03 | 0.997 | 282.1318 | 3.9E−04 | 0.997 | 18.4% | 17.1% | 93.0% | 50 | 5000 |
| Indole-3-Lactic acid | 311.1390 | 4.2E−03 | 0.995 | 312.1424 | 8.1E−04 | 0.995 | 19.5% | 19.4% | 99.6% | 50 | 5000 |
| Indole-3-Propionic acid | 295.1441 | 2.1E−03 | 0.996 | 296.1475 | 3.8E−04 | 0.997 | 19.5% | 18.1% | 92.8% | 50 | 5000 |
| Phenylacetic acid | 242.1176 | 5.0E−03 | 0.999 | 243.1209 | 8.0E−04 | 0.994 | 16.2% | 16.2% | 99.7% | 30 | 3000 |
| Phenyllactic acid | 272.1281 | 3.0E−03 | 0.999 | 273.1315 | 5.0E−04 | 0.998 | 17.3% | 16.4% | 94.6% | 50 | 5000 |
| Phenylpyruvic acid | 375.1703 | 6.1E−03 | 0.996 | 376.1737 | 1.4E−03 | 0.994 | 24.9% | 23.7% | 95.4% | 36 | 3600 |
| 4-Hydroxyphenylacetic acid | 258.1125 | 4.1E−03 | 0.999 | 259.1158 | 6.2E−04 | 0.981 | 16.2% | 14.9% | 92.0% | 30 | 3000 |
| 4-Hydroxyphenyllactic acid | 288.1230 | 5.1E−03 | 0.999 | 289.1264 | 8.1E−04 | 0.999 | 17.3% | 16.1% | 92.7% | 50 | 5000 |
| 4-Hydroxyphenylpyruvic acid | 391.1652 | 3.7E−03 | 0.994 | 392.1686 | 8.3E−04 | 0.989 | 24.9% | 22.7% | 91.1% | 36 | 3600 |

TABLE 5

| | Calibration Carve | | | | | | | | | |
|---|---|---|---|---|---|---|---|---|---|---|
| Analyte | STD-1 | STD-2 | STD-3 | STD-4 | STD-5 | STD-6 | STD-7 | STD-S | STD-9 | STD-10 |
| Formic acid | 806.22 | 1343.49 | 2239.49 | 3732.48 | 6220.8 | 10368 | 17280 | 28800 | 48000 | 80000 |
| Butyric acid | 50.39 | 83.98 | 139.97 | 233.28 | 388.8 | 648 | 1080 | 1800 | 3000 | 5000 |
| Isobutyric acid | 50.39 | 83.98 | 139.97 | 233.28 | 388.8 | 648 | 1080 | 1800 | 3000 | 5000 |
| Isovaleric acid | 50.39 | 83.98 | 139.97 | 233.28 | 388.8 | 648 | 1080 | 1800 | 3000 | 5000 |
| Caproic acid | 90.7 | 151.17 | 251.94 | 419.9 | 699.8 | 1166 | 1944 | 3240 | 5400 | 9000 |
| 2-Methylbutyric acid | 50.39 | 83.98 | 139.97 | 233.28 | 388.8 | 648 | 1080 | 1800 | 3000 | 5000 |
| 2-Hydroxybutyric acid | 50.39 | 83.98 | 139.97 | 233.28 | 388.8 | 648 | 1080 | 1800 | 3000 | 5000 |
| 3-Hydroxylbutyric acid | 50.39 | 83.98 | 139.97 | 233.28 | 388.8 | 648 | 1080 | 1800 | 3000 | 5000 |
| 2-Hydroxyisovaleric acid | 50.39 | 83.98 | 139.97 | 233.28 | 388.8 | 648 | 1080 | 1800 | 3000 | 5000 |
| 2-Hydroxyisocaproic acid | 50.39 | 83.98 | 139.97 | 233.28 | 388.8 | 648 | 1080 | 1800 | 3000 | 5000 |
| alpha-Ketoisovaleric acid | 50.39 | 83.98 | 139.97 | 233.28 | 388.8 | 648 | 1080 | 1800 | 3000 | 5000 |
| alpha-Ketoisocaproic acid | 50.39 | 83.98 | 139.97 | 233.28 | 388.8 | 648 | 1080 | 1800 | 3000 | 5000 |
| Indole-3-Acetic acid | 50.39 | 83.98 | 139.97 | 233.28 | 388.8 | 648 | 1080 | 1800 | 3000 | 5000 |
| Indole-3-Lactic acid | 50.39 | 83.98 | 139.97 | 233.28 | 388.8 | 648 | 1080 | 1800 | 3000 | 5000 |
| Indole-3-Propionic acid | 50.39 | 83.98 | 139.97 | 233.28 | 388.8 | 648 | 1080 | 1800 | 3000 | 5000 |
| Phenylacetic acid | 30.23 | 50.39 | 83.98 | 139.97 | 233.3 | 389 | 648 | 1080 | 1800 | 3000 |
| Phenyllactic acid | 50.39 | 83.98 | 139.97 | 233.28 | 388.8 | 648 | 1080 | 1800 | 3000 | 5000 |
| Phenylpyruvic acid | 36.28 | 60.47 | 100.78 | 167.96 | 279.9 | 467 | 778 | 1296 | 2160 | 3600 |
| 4-Hydroxyphenylacetic acid | 30.23 | 50.39 | 83.98 | 139.97 | 233.3 | 389 | 648 | 1080 | 1800 | 3000 |
| 4-Hydroxyphenyllactic acid | 50.39 | 83.98 | 139.97 | 233.28 | 388.8 | 648 | 1080 | 1800 | 3000 | 5000 |
| 4-Hydroxyphenylpyruvic acid | 36.28 | 60.47 | 100.78 | 167.96 | 279.9 | 467 | 778 | 1296 | 2160 | 3600 |

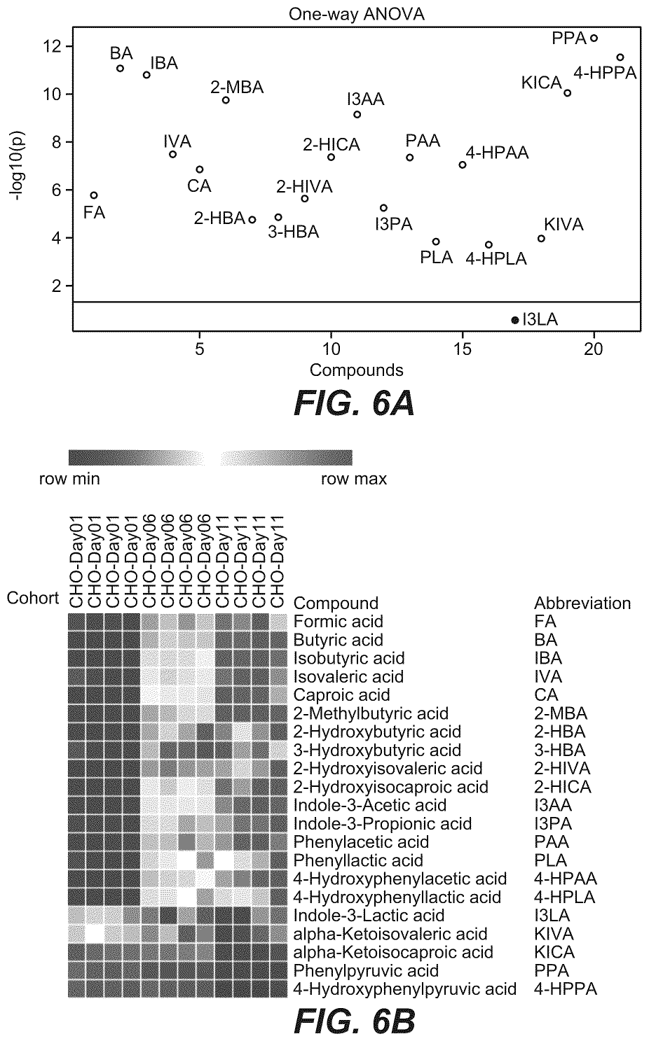

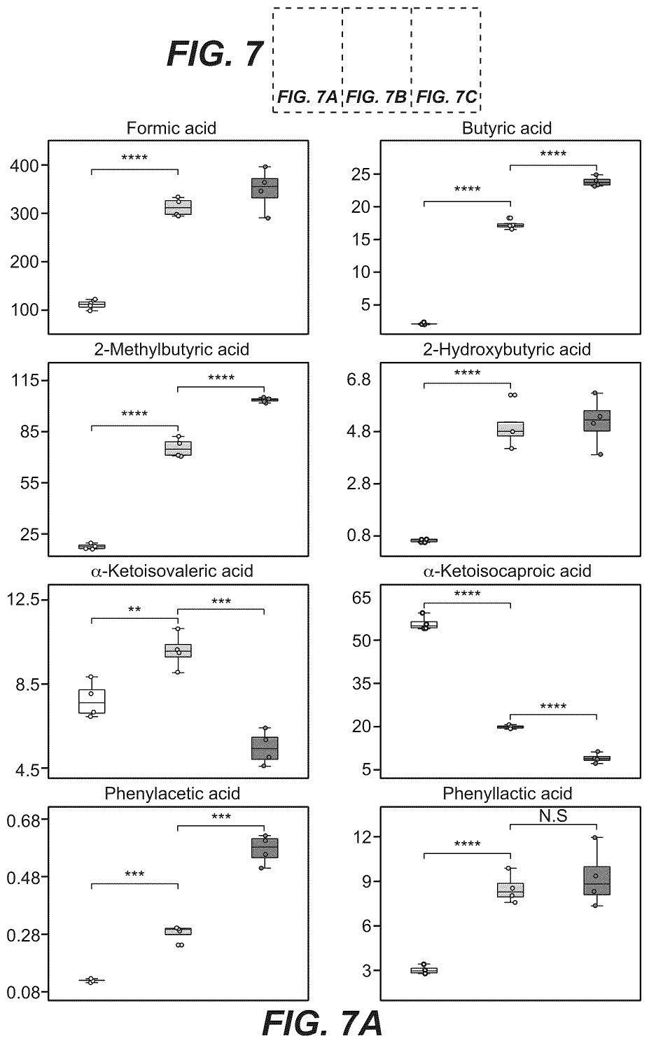

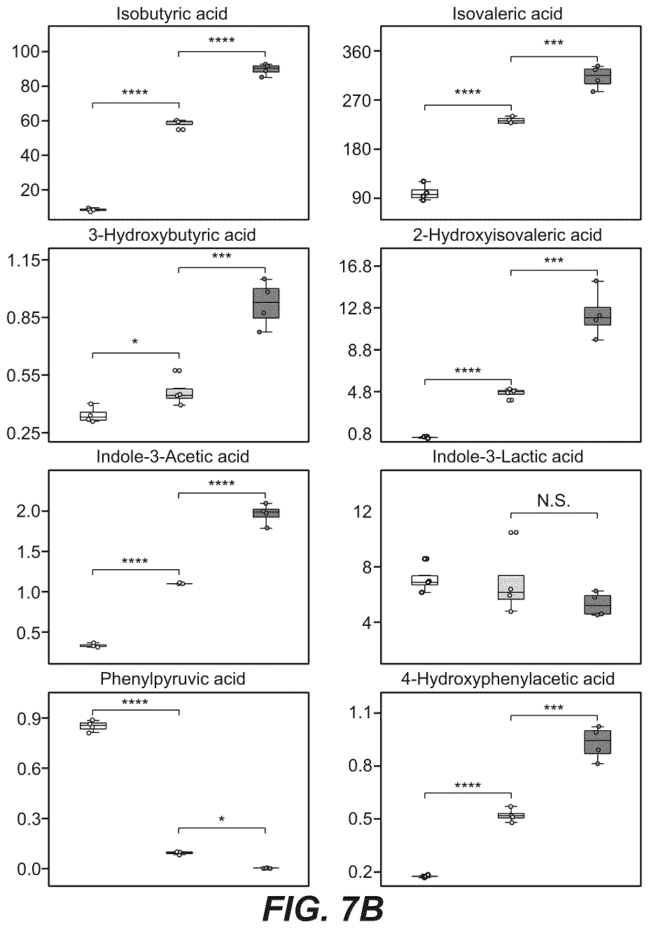

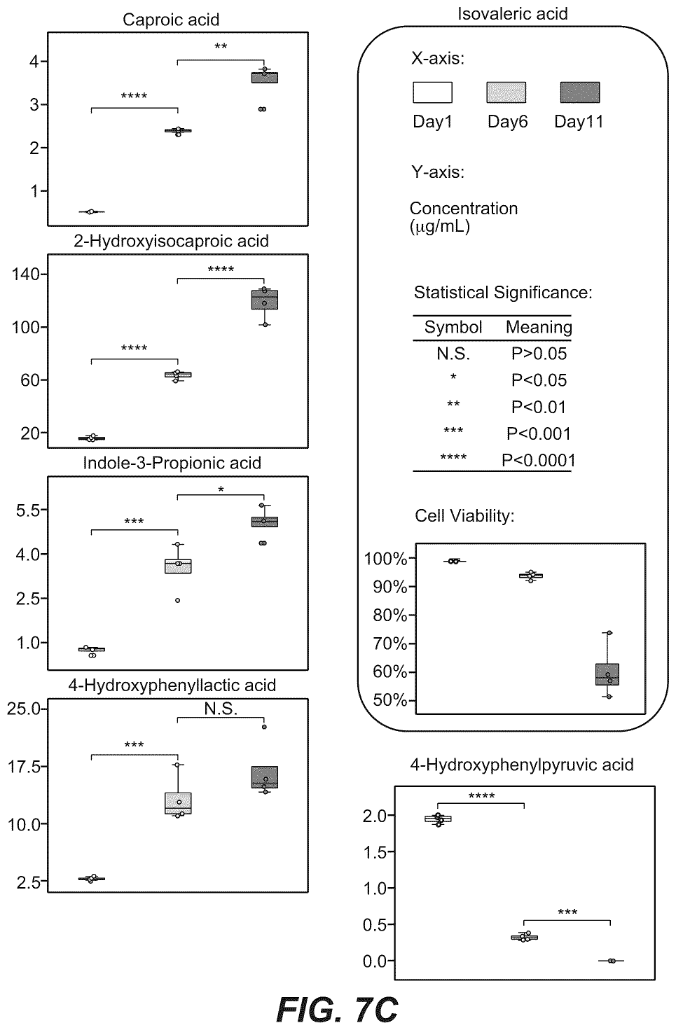

Statistical Significance of Quantification. Statistical significances associated with the whole production process involving three group comparisons are presented in FIG. 6B to summarize the p-values computed by one-way ANOVA (Analysis of Variance). Global targeted view of metabolic variations is visualized in FIG. 6B to illustrate the relative fold changes generated by Heat Map analysis. Briefly, 20 metabolites of total 21 target analytes demonstrated statistically significant (p<0.05) differences with p-values ranging from 1.9×E−4 to 9.2×E−12 when comparing three cellular stages in the mAb production process. Interestingly, 80% of the significantly altered metabolites indicated consecutive increase from day 1 growth phase, to day 6 stationary phase, and until day 11 cell death phase. Isovaleric acid is believed to adversely impact cell growth at concentrations of 1 mM (102 μg/mL) and beyond. The targeted metabolomics data in this study showed that the level of isovaleric acid already reached 2-3 mM during the middle to late production stage of CHO cell fed-batch bioprocess, demonstrating that the level of isovaleric acid reached undesirable levels. We have also demonstrated that the present methods may also be used for rapid and simultaneous quantification of other inhibitory metabolites, such as isobutyric acid and 2-methylbutyric acid. In addition, in the same experiment we have also proposed several new growth inhibition related metabolites and quantitatively explored their metabolic responses in production cell cultures for the first time, including 2-hydroxyisovaleric acid and 2-hydroxyisocaproic acid showing obviously negative correlation with cell viability (FIG. 7), but alpha-ketoisovaleric acid and alpha-ketoisocaproic acid exhibiting very similar trend to cell activity especially from stationary phase to death phase (FIG. 7). As demonstrated through FIG. 6, the present methods can provide comprehensive illustration of a broad range of metabolite alterations could provide a global perspective to differentiate cellular stages and metabolic behaviours.

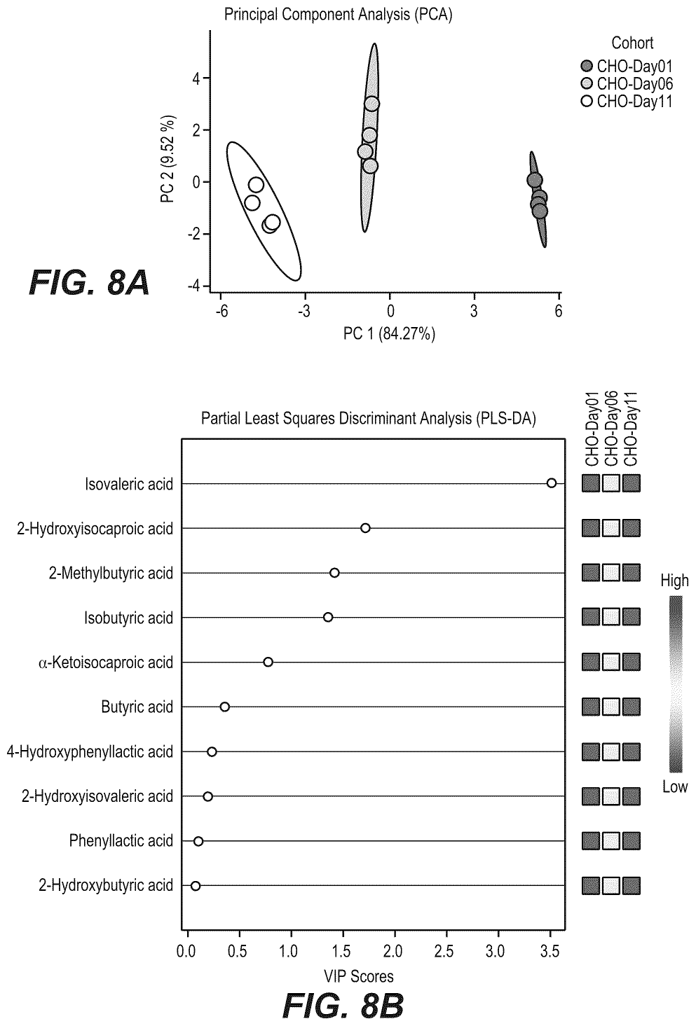

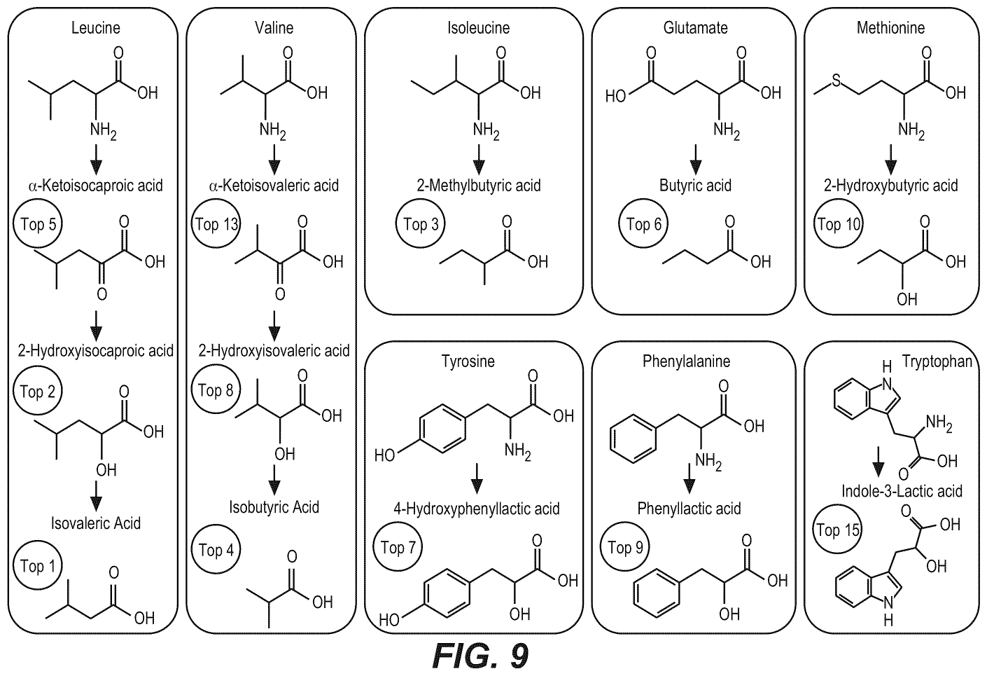

Multivariate Analysis with PCA/PLS-DA. We further conducted multivariate statistical analysis involving Principal Component Analysis (PCA) and Partial Least Squares Discriminant Analysis (PLS-DA). Essentially, multivariate PCA (unsupervised approach) and PLS-DA (supervised approach) are orthogonal projection techniques for dimensionality reduction of complex dataset in order to elucidate the intrinsic classification and feature selection in comprehensive metadata. As shown in FIG. 8A, the compact clustering of CHO cell fed-batch culture samples from four individual bioreactors, combined with clear group separation between different time points, indicated that production cell culture samples were well distinguished by cellular stages through PCA analysis. Data structure and quality were explored, suggesting no analytical or biological outliers were required to be excluded from the dataset. As expected, CHO cell fed-batch cultures from independent bioreactors in mAb production process were more consistent on day 1 during initial growth phase (FIG. 8A, red cluster), while small extent of differentiation occurred on day 11 during final death phase (FIG. 8A, blue cluster) owing to cell viabilities were decreasing with distinct speeds in this stage (FIG. 7). Moreover, as presented in FIG. 8B, top 10 metabolic features contributing to the classification model were selected based on PLS-DA analysis. The VIP (Variable Importance in Projection) score of each metabolite was calculated as a weighted sum of the squared correlations between original variable and integrated PLS-DA components, providing a quantitative assessment for the discriminatory power of each metabolic feature. Next, amino acid metabolic sources for the growth inhibition related metabolites with top VIP scores in PLS-DA analysis are provided in FIG. 9 together with their chemical structures. Strikingly, top 5 metabolic features were all produced from branched-chain amino acids (BCAAs) including valine, leucine, and isoleucine. More importantly, isovaleric acid ranking as top 1, 2-hydroxyisocaproic acid ranking as top 2, and alpha-ketoisocaproic acid ranking as top 5 were specifically generated from leucine catabolism. Meanwhile, aromatic lactic acids derived from phenylalanine, tyrosine and tryptophan pathways appeared to be more critical and significantly more intense than other related small molecule byproducts such as aromatic acetic acids and aromatic pyruvic acids. Tremendous efforts have been dedicated to understand and control of those growth inhibition related metabolites in CHO cell fed-batch cultures, our targeted metabolomics platform through absolute quantification and multivariate statistical analysis proved to be a powerful tool for actively tracking and advancing this process.

### Example 4: Relative Quantification

### Cell Culture

High seeding density fed-batch production cultures were performed in shake flasks with proprietary chemically defined production media. For N−1 inoculum train, cells were seeded at 2×106 cells/ml on Day 0 and maintained for 7 days with feed media and glucose addition on day 4. On day 2, 4 and 7 of the N−1 culture, inoculum train cells were seeded into the production cultures by either complete or partial media exchange (1:1 or 3:1 fresh media to spent media ratio) to potentially mimic fully or partially perfused cultures, setting up production culture with 3×107 cells/mL starting cell density on Day 0. The short 3 days production run was set up to illustrate the effects of inhibitory molecules on cell growth obtained from N−1 cultures, where overtime cells were exposed to increasingly higher concentrations of the accumulated inhibitory molecules. The intensified shake flask production process mimics the intensified N process in bioreactors, while the complete or partial media exchange used for setting up the production culture was used to mimic different perfusion speed in N−1.

### Sample Collection

For sample collection, cell culture samples were acquired at interested time points. Collected samples were centrifuged to separate the cell pellets and supernatants. CHO cell fed-batch culture supernatants were transferred into clean tubes and submitted for further metabolomics analysis.

### Metabolite Extraction and Derivatization

Metabolites of interest were extracted from fed-batch culture samples using cold acetonitrile; and then derivatized with 10 uL of 0.3 M O-BHA (in MeOH) and 10 uL of 0.3 M EDC (in MeOH) at 800 RPM and 25° C. for 10 minutes. Subsequently, 50 uL of water and 400 uL of dichloromethane were added to perform liquid-liquid phase separation. Samples were mixed and centrifuged again. Organic layer aliquots containing derivatized compounds were transferred, dried, reconstituted in 100 uL of water, and submitted for further LC-HRMS analysis.

### LC-HRMS Analysis

LC-HRMS experiments were conducted using the setup described in Example 3. For data analysis, LC-HRMS peak area of each targeted analyte was obtained as relative quantification to increase the throughput of inhibitory metabolites monitoring. LC-HRMS peak area of initial time point (e.g. Day 2 in N−1 stage of this example) can be utilized as baseline to evaluate the accumulation of inhibitory metabolites during cell culture bioprocesses.

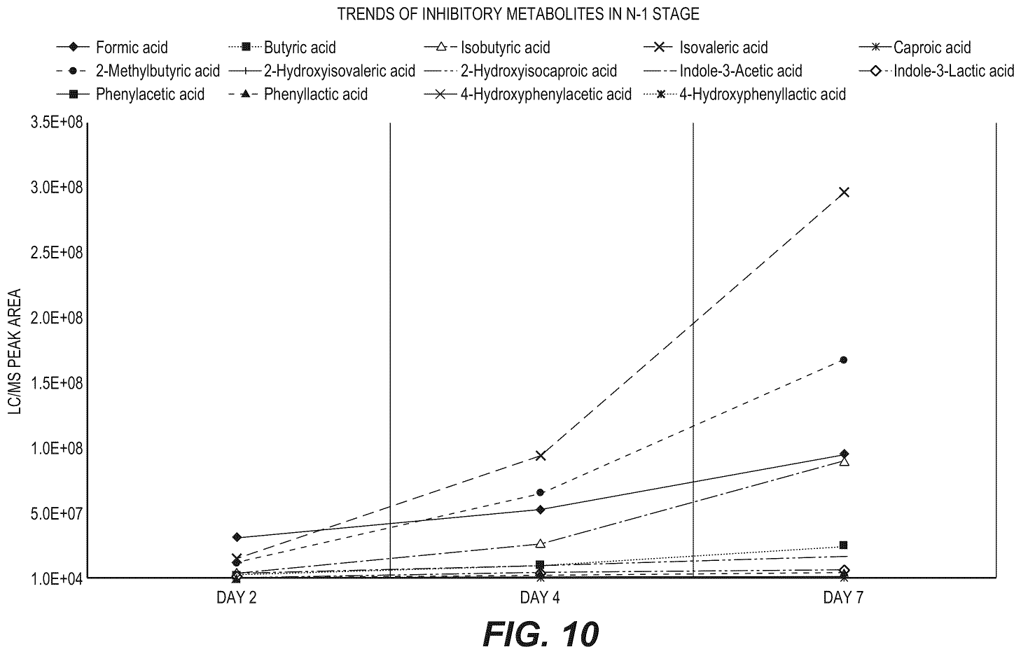

The results for relative quantification of multiple metabolites are indicated in Table 6. FIG. 10 provides a plot of this data, demonstrating the trends for each of the metabolites. In addition to providing relative quantification of metabolites, it is also possible to provide relative quantification at the same time of one or more of the cell culture components. This allows for further relative quantification. Such relative analysis may be helpful when monitoring the progress of or optimising a cell culture.

TABLE 6

| Analyte | Day 2 | Day 4 | Day 7 |
|---|---|---|---|
| Formic acid | 3.2E+07 | 5.3E+07 | 9.6E+07 |
| Butyric acid | 2.8E+06 | 9.8E+06 | 2.5E+07 |
| Isobutyric acid | 4.7E+06 | 2.8E+07 | 9.0E+07 |
| Isovaleric acid | 1.6E+07 | 9.5E+07 | 3.0E+08 |
| Caproic acid | 2.9E+05 | 6.6E+05 | 1.4E+06 |
| 2-Methylbutyric acid | 1.3E+07 | 6.6E+07 | 1.7E+08 |
| 2-Hydroxyisovaleric acid | 1.7E+05 | 4.2E+05 | 7.7E+05 |
| 2-Hydroxyisocaproic acid | 4.3E+06 | 1.1E+07 | 1.7E+07 |
| Indole-3-Acetic acid | 2.9E+05 | 6.0E+05 | 1.0E+06 |
| Indole-3-Lactic acid | 5.1E+05 | 4.8E+06 | 7.1E+06 |
| Phenylacetic acid | 1.5E+05 | 4.0E+05 | 8.3E+05 |
| Phenyllactic acid | 3.9E+05 | 3.0E+06 | 5.1E+06 |
| 4-Hydroxyphenylacetic acid | 9.9E+04 | 1.6E+05 | 2.5E+05 |
| 4-Hydroxyphenyllactic acid | 2.4E+05 | 1.3E+06 | 2.1E+06 |

1. A method for quantifying the concentration of multiple metabolites in a sample of a cell culture, wherein each metabolite comprises at least one carbonyl group, the method comprising:
(a) adding a known amount of an internal standard corresponding to each of the multiple analytes of the sample;
(b) contacting the sample with a reagent comprising a carbonyl reactive group and a masking group, thereby derivatising the carbonyl group of each metabolite with the masking group and providing a derivatised sample;
(c) subjecting the derivatised sample to chromatographic separation and mass spectrometry; and
(d) quantifying the amount of each of the multiple metabolites based on a monoisotopic signal obtained for said metabolite,
wherein said quantifying comprises comparing the monoisotopic signal obtained for each said one of the multiple metabolites to an external calibration for said one of the multiple metabolites, after normalising the external calibration using the signal obtained for the known amount of the stable isotope labelled analogue corresponding to the said one of the multiple metabolites;
wherein the monoisotopic signal obtained for said metabolite corresponds to the most abundant positive ion isotopologue, unless said most abundant positive ion isotopologue has a signal at or above the upper limit of the external calibration curve for said metabolite, in which case the monoisotopic signal obtained for said metabolite corresponds to a less abundant positive ion isotopologue of said metabolite;
wherein the sample is a sample of cell culture medium of the cell culture, and/or of cell lysate of the cell culture; and
wherein the cell culture is a mammalian cell culture, insect cell culture, yeast cell culture, or prokaryotic cell culture.

2. (canceled)

3. (canceled)

4. The method of claim 1, wherein the cell culture is a Chinese Hamster Ovary (CHO) cell culture, optionally a CHO K1 cell culture, a CHO K1SV cell culture, a DG44 cell culture, a DUKXB-11 cell culture, a CHOK1S cell culture, or a CHO K1M cell culture, or a targeted integration engineered CHO or CHO derivative cell culture.

5. The method of claim 1, wherein the method further comprises on-line sampling of the cell culture to obtain the sample.

6. The method of claim 5, wherein said quantifying is completed within about 4 hours of on-line sampling of the cell culture.

7. The method of claim 6, wherein said quantifying is completed within about 2 hour of on-line sampling of the cell culture.

8. The method of claim 1, wherein each internal standard corresponds to an isotopologue of at least two nominal mass units more than the most abundant isotopologue of one of the multiple analytes.

9. The method of claim 1, wherein each internal standard comprises an isotopologue of one of the multiple metabolites having a double, triple, or quadruple isotopic label, wherein each isotopic label is selected from a deuterium, a carbon-13 (¹³C), or nitrogen-15 (¹⁵N).

10. The method of claim 1, wherein one or more of the multiple metabolites are inhibitory metabolites; or comprise of one or more amino acid derived metabolites.

11. (canceled)

12. The method of claim 1, wherein the multiple metabolites comprise or consist of one or more of formic acid, butyric acid, isobutyric acid, isovaleric acid, caproic acid, 2-methylbutyric acid, 2-hydroxybutryic acid, 3-hydroxybutyric acid, 2-hydroxyisovaleric acid, 2-hydroxyisocaproic acid, α-ketoisovaleric acid, α-ketoisocaprioc acid, indole-3-acetic acid, indole-3-lactic acid, indole-3-propionic acid, phenylacetic acid, phenyllactic acid, phenylpyruvic acid, 4-hydroxyphenylacetic acid, 4-hydroxyphenyllactic acid, 4-hydroxyphenylpyruvic acid, valine, leucine, isoleucine, aspartic acid, tryptophan, malate, fumarate, succinate, α-ketoglutaric acid.

13. (canceled)

14. The method of claim 12, wherein the internal standard corresponding to each of the multiple metabolites is:
butyric acid-D2 when butyric acid is a said metabolite;
isobutyric acid-D3 when isobutyric acid and/or formic acid are said metabolites;
isovaleric acid-D2 when isovaleric acid and/or 2-methylbutyric acid are said metabolites;
caproic acid-D2 when caproic acid is a said metabolite;
3-hydroxybutyric acid-D4 when 2-hydroxybutyric acid and/or 3-hydroxybutyric acid and/or 2-hydroxyisovaleric acid and/or 2-hydroxyisocaproic acid are said metabolites;
α-ketoisocaproic acid-D3 when α-ketoisovaleric acid and/or α-ketoisocaproic acid are said metabolites;
indole-3-acetic acid-D2 when indole-3-acetic acid and/or indole-3-lactic acid and/or indole-3-propionic acid are said metabolites;
phenyllactic acid-D3 when phenylacetic acid and/or phenyllactic acid and/or phenylpyruvic acid are said metabolites; and
4-hydroxyphenyllactic acid-D3 when 4-hydroxyphenylacetic acid and/or 4-hydroxyphenyllactic acid and/or 4-hydroxyphenylpyruvic acid are said metabolites.

15. The method of claim 1, wherein the step (b) of contacting further comprises activating the carbonyl group of each metabolite, thereby providing an activated carbonyl group on each metabolite for reaction with the carbonyl reactive group.

16. The method of claim 15, wherein activating the carbonyl group of each metabolite comprises contacting the sample with N−(3-dimethylaminopropyl)-N′-ethylcarbodiimide (EDC) or a salt thereof, such as EDC hydrochloride.

17. (canceled)

18. (canceled)

19. The method of claim 1, wherein the reagent comprising the carbonyl reactive group and the masking group is O-benzylhydroxylamine (0-BHA), or a salt thereof, such as O-BHA hydrochloride.

20. (canceled)

21. (canceled)

22. The method of claim 1, wherein the mass spectrometry comprises introducing the eluate from the chromatographic separation into a mass spectrometer ion source to generate positive ions of analyte molecules and performing and obtaining full scan mass spectra on an accurate mass high resolution mass spectrometer.

23. (canceled)

24. (canceled)

25. The method of claim 1, wherein the full scan accurate mass high resolution mass spectrometry comprises scanning a mass to charge ratio of from at least about 50 to not more than about 800 m/z;
optionally wherein the full scan accurate mass high resolution mass spectrometry comprises scanning a mass to charge ratio of from about 100 to about 500 m/z.

26. The method of claim 1, wherein the mass accuracy of the mass spectrometry is 20 ppm or better, and wherein the resolution of the mass spectrometry is at least 35,000.

27. (canceled)

28. The method of claim 1, wherein the dynamic range for quantifying the concentration of each of the multiple metabolites is at least 2.5 orders of magnitude.

29. (canceled)

30. A method for relative quantification of multiple metabolites in a sample, wherein each metabolite comprises at least one carbonyl group, the method comprising
contacting the sample with a reagent comprising a carbonyl reactive group and a masking group, thereby derivatising the carbonyl group of each metabolite with the masking group and providing a derivatised sample;
subjecting the derivatised sample to chromatographic separation and full scan accurate mass high resolution mass spectrometry; and
quantifying the amount of each of the multiple metabolites based on a monoisotopic signal obtained for each said metabolite,
wherein said quantifying comprises comparing the monoisotopic signal obtained for each said one of the multiple metabolites to the monoisotopic signal obtained for each said other of the multiple metabolites, thereby obtaining the relative quantification,
wherein the monoisotopic signal obtained for each one of the metabolites corresponds to the most abundant positive ion isotopologue, unless said most abundant positive ion isotopologue has a signal at or above the upper limit of quantification for said metabolite, in which case the monoisotopic signal obtained for said metabolite corresponds to a less abundant positive ion isotopologue of said metabolite.

31. A method of monitoring the course of a cell culture, comprising:
(a) performing the method of claim 1 at a first time point, wherein the sample is a sample of a cell culture;
(b) repeating the method of claim 1 at a second time point; and
(c) optionally repeating the method of claim 1 at one or more subsequent time points,
wherein the monitoring comprises comparing the levels of each of the multiple metabolites between each of the multiple time points.

32. (canceled)

33. (canceled)

34. A method of optimising a cell culture, comprising:
(a) performing the method of claim 1 at a first time point, wherein the sample is a sample of a cell culture;
(b) repeating the method of claim 1 at a second time point; and
(c) optionally repeating the method of claim 1 at one or more subsequent time points,
and, in response to a change in the concentration of at least one of the multiple metabolites between the first time point and a second or subsequent time point, adjusting a cell culture condition.

35. The method of claim 34, wherein the change in concentration comprises an increase in concentration of at least one of the multiple metabolites between the first time point and a second or subsequent time point, optionally wherein the said at least one of the multiple metabolites is an inhibitor.

36. The method of claim 34, wherein the cell culture condition is selected from perfusion rate, temperature, pH, dissolved oxygen (dO₂), cell culture duration, and the level of one or more cell culture ingredient(s).

37.-42. (canceled)
